(19) United States
(12) Patent Application Publication
(10) Pub. No.: US 2024/0050458 A1
(43) Pub. Date: Feb. 15, 2024

(54) Compositions And Methods For Inducing Apopotosis In Anaerobic Cells And Related Clinical Methods For Treating Cancer And Pathogenic Infections

(71) Applicant: Suzannah Jane ROBERTS, Kirkland, WA (US)

(72) Inventor: Suzannah Jane ROBERTS, Kirkland, WA (US)

(21) Appl. No.: 18/380,612

(22) Filed: Oct. 16, 2023

Related U.S. Application Data
(63) Continuation of application No. 18/094,729, filed on Jan. 9, 2023, now abandoned.
Application No. 18/094,729 is a continuation of application No. 17/831,446, filed on Jun. 3, 2022, now abandoned.
Application No. 17/831,446 is a continuation of application No. 17/509,127, filed on Oct. 25, 2021, now abandoned.
Application No. 17/509,127 is a continuation of application No. 17/200,895, filed on Mar. 14, 2021, now abandoned.
Application No. 17/200,895 is a continuation of application No. 16/636,898, filed on Feb. 5, 2020, now abandoned.
(60) Provisional application No. 62/605,352, filed on Aug. 7, 2017.

(51) Int. Cl. A61K 31/7048; A61P 35/00; A61K 9/00; A61K 31/7034; A61K 45/06

(52) U.S. Cl. CPC A61K 31/7048; A61P 35/00; A61K 9/0019; A61K 9/0053; A61K 31/7034; A61K 45/06

(57) ABSTRACT

The invention provides potent anti-cancer methods and compositions that employ novel glycome compounds exemplified by glyco-benzaldehydes that disrupt anaerobic respiration and trigger apoptosis in cancer cells. Additionally, the invention provides useful compositions and methods to treat viral and microbial infections, and for enhancing suppressed immune systems, including by disrupting alpha-N-acetylgalactosaminidase (nagalase) function and lowering circulating nagalase blood levels. In certain anti-cancer and immune enhancing methods and compositions of the invention glyco-benzaldehyde compounds, such as the plant-derived glyco-benzaldehyde helicidum, are employed, alone or in combination, to potently destroy tumors and circulating cancer cells, and significantly prolong survival of cancer patients, including treatment-resistant Stage III and Stage IV cancer patients.

### RELATED APPLICATIONS

This patent application claims the benefit of priority from U.S. Provisional patent application No. 62/605,352, filed Aug. 7, 2017, which is incorporated by reference herein in its entirety for all purposes.

### BACKGROUND

Cancer is the second leading cause of death in the United States and other developed nations. The US National Cancer Institute (NCI) reported 8.2 million cancer-related deaths worldwide in 2012, and 14.1 million new cases diagnosed that year. New cancer diagnoses globally will rise to approximately 24 million by 2030. According to current NCI statistics, there will be an estimated 1,735,350 new cases of cancer diagnosed and 609,640 cancer deaths in the US in 2018.

The economic burdens of diagnosing and treating cancer on healthcare systems around the world are enormous, with estimated national expenses for cancer care in the United States in 2017 approaching $150 billion. In future years, costs will continue to as mean population age and cancer prevalence increase, and more expensive treatments are adopted as standards of care.

Conventional treatments for cancer typically involve a combination of surgery, chemotherapy, radiation and hormonal therapy to eradicate neoplastic cells in a patient. All of these treatment modalities impose significant morbidity and added risks, for example increased risks of infection and many other adverse health conditions that attend the rigors of cancer treatment.

Despite considerable advances in detection and treatment of cancer over the past several decades, conventional treatments like surgery, chemotherapy and radiation often achieve only modest improvements in survival, while imposing significant adverse impacts on quality of life, raising questions about cost-effectiveness and overall clinical benefits of such treatments.

In view of the foregoing there is a compelling need in the medical arts for alternative tools and methods to prevent, treat and clinically manage proliferative disorders, including cancer.

A related need exists for therapeutic compositions and methods to treat viral and other pathogenic infections, which may be associated with cancer, suppression of immune functions attending cancer, or arise independently.

It is therefore an object of the present invention to provide novel methods and compositions for treating and preventing cellular proliferative disorders, including cancer.

It is a further object of the invention to provide novel methods and compositions for immunotherapy to treat viral and other infections, including infections that occur in association with cancer.

It is an additional object of the present invention to provide novel methods and compositions for the treatment of resistant forms of cellular proliferative disorders, including, but not limited to, stage IV or terminal cancers

It is yet another object of the present invention to provide effective treatment and management tools to increase quality of life and survival in cancer patients, including advanced (e.g., Stage III and Stage IV) cancer patients, and “refractory” or “resistant” cancer patients who have not found effective treatment through conventional onotherapies (surgery, chemotherapy, radiation, hormonal oncotherapy).

### SUMMARY OF EXEMPLARY EMBODIMENTS OF THE INVENTION

The invention achieves the foregoing objects and satisfies additional objects and advantages by providing novel and surprisingly effective anti-cancer and immunotherapeutic compositions and methods for use in mammalian subjects, including veterinary and human clinical subjects.

In one aspect, the invention provides compositions and methods for inducing apoptosis in a circulating tumor cell (CTC) population, cancerous tissue or cancerous tumor in a mammalian subject, sufficient to prevent progression of a cancer disease condition or symptom(s) in the subject. These novel methods and tools focus on administering a cancer apoptosis-inducing effective amount of Salicinium®, exemplified by one or more glyco-benzaldehyde compound(s), in an oncotherapeutic treatment protocol that is demonstrated herein to potently induce apoptosis in the targeted CTC population, cancer tissue or tumor in the subject. According to these therapeutic methods, after two-six months of treatment the subject's cancer disease condition is most frequently stabilized (marked by no detectable increase in the CTC population, no increase in the number or size of cancerous tumors, and no new metastases of cancer cells to form new tumors). In other cases, the subject's cancer condition is substantially alleviated to a state of “partial remission” (marked by a reduction in CTCs, tumor number, tumor size, cancer blood markers or other diagnostic indicia of disease abatement), or is eradicated to a state of “complete remission” (marked by no tumors, CTCs, or cancer blood markers detectable by any conventional cancer diagnostic method). For all cancer types, use of apoptosis-inducing Salicinium methods and compositions results in statistically significant increase in five-year survival rates among subjects including treatment-resistant and non-responsive Stage IV cancer subjects.

In related aspects of the invention methods and compositions are provided for treating Stage IV cancer in mammalian subjects. These methods involve administering an effective amount of a Salicinium® compound (exemplified by glyco-benzaldehyde compounds) to destroy cancer cells by a mechanism of apoptosis, in sufficient numbers to ablate circulating cancer cells and reduce or eliminate tumors in the subject over an effective treatment period, thereby extending long-term survival of the subject.

In additional aspects, the foregoing methods extend a five-year survival rate among Stage IV cancer patients (compared to median survival within a comparable group of conventionally-treated or untreated cancer patients) by at least 15%. In certain embodiments, five-year survival rate among Stage IV cancer patients (compared to median survival within a comparable group of conventionally-treated or untreated cancer patients) by at least 25%-50%, 50%-100%, and in certain cases by as much as two-fold, three-fold, four-fold or even five-fold or greater.

Additionally, the invention provides diverse methods of immunotherapy, including immunotherapy involving modulation of alpha-N-acetylgalactosaminidase (nagalase) physiology and circulating nagalase blood levels). Salicinium® is administered to mammalian subjects diagnosed with, or determined to be at risk of developing, cancer or a viral or microbial infection (typically determined by the subject having above-normal nagalase levels in a blood sample). This anti-nagalase treatment involves potent interference by Salicinium with nagalase synthesis and/or activity. By this mechanism, Salicinium effectively reduces or shuts down nagalase production in cancer cells and cells infected with viruses. This potentiates activation of the subject's immune response, previously suppressed by elevated nagalase expression.

In related methods the invention provides for active immunotherapy targeting cancer and viral-infected cells for destruction by the subject's own immune system. An anti-nagalase effective amount of Salicinium® is administered to reduce nagalase levels, resulting in clearance of immunosuppressive nagalase from the subject's blood and tissues, allowing in turn for activation of immune effector cells (e.g., monocytes/macrophages and downstream T cells, natural killer (NK) cells, and B-cells) to fight cancer and viral-infected cells (along with other pathogens and disease conditions that result in aberrant high nagalase levels in the blood.

Immunotherapy methods of the invention can be adapted to treat any proliferative disorder, including any cancer, hyperplastic conditions, autoimmune disorders, psoriasis, and a wide range of pathogenic infections, particularly viral infections (including influenza, and retroviruses like herpesviruses, Epstein-Barr virus (EBV), human T-cell leukemia virus-1 (HTLV-1) (causative agent of adult T-cell leukemia/lymphoma (ATL)), Raus sarcoma virus, Human papilloma viruses (HPVs), and Human Immunodeficiency Virus (HIV), among others).

### BRIEF DESCRIPTION OF THE DRAWINGS

The objects, features, aspects and advantages of the invention will be apparent to the skilled artisan from the Detailed Description below.

FIG. 1 graphically illustrates Salicinium® induction of apoptosis in circulating tumor cells (CTCs). Each bar depicts the percentage of cultured CTC cells from patient samples grouped by primary cancer types exhibiting apoptosis within 24 hours following a single exposure to an anti-cancer effective dose of Salicinium.

FIG. 2 graphically depicts results of a five-year disease progression and survival study for groups of breast, colon, lung and prostate cancer patients receiving anti-cancer treatment with Salicinium® (in side-by-side comparison to published median survival statistics for all patients diagnosed with the indicated Stage IV cancer, treated and untreated). Patients diagnosed with Stage IV breast, colon, lung and prostate cancer were administered Salicinium comprising helicidum according to the protocols described herein, and the percentages of enrolled patients surviving are shown (left axis=surviving percentage of original patient population in each group). These surviving subjects were evaluated at the end of the study and determined to be either in stable disease, partial remission, or complete remission (no cancer symptoms detectable by conventional methods, including tumor visualization, cytology, cancer markers in blood, etc.), as further detailed in Table 1.

FIG. 3 graphically depicts the results of a five-year survival study for ovarian, pancreatic, and melanoma cancer patients receiving anti-cancer treatment employing Salicinium® (in side-by-side comparison to published median survival statistics for all patients diagnosed with the indicated Stage IV cancer). Patients diagnosed with Stage IV ovarian, pancreatic and melanoma cancer were administered Salicinium comprising helicidum according to the protocols described herein, and the percentages of enrolled patients surviving are shown (left axis=surviving percentage of original patient population in each group). These surviving subjects were evaluated at the end of the study and determined to be either in stable disease, partial remission, or complete remission (no cancer symptoms detectable by conventional methods, including tumor visualization, cytology, cancer markers in blood, etc.), as further detailed in Table 1.

FIG. 4 is a graphic representation of assay results measuring Salicinium®-mediated increases in NK cell cancer-killing activity, using immune cells harvested from cancer patients (presumptively immunosuppressed) activated against cancer cells in culture.

### DETAILED DESCRIPTION OF EXEMPLARY EMBODIMENTS OF THE INVENTION

The instant invention provides novel methods and compositions for treating and clinically managing cancer, and additional methods for treating and managing viral infections and other pathogen-mediated diseases in mammalian subjects. Long-term clinical studies are presented below involving many hundreds of human clinical subjects diagnosed at the outset of treatment with stage IV cancer (often treatment-resistant or non-responsive stage IV cancer, refractory to prior, extensive conventional oncotherapy), which demonstrate dramatically increased survival rates of subjects treated with novel Salicinium® compositions and methods described herein.

In exemplary studies, stage IV cancer patients presenting with a diverse array of cancers benefitted from cancer therapy employing a proprietary drug formulation of Salicinium, comprising a glyco-benzaldehyde as further described below. One exemplary form of Salicinium is represented by the formula I below, depicting a natural glyco-benzaldehyde helicidum. In related examples the sugar or “glycome” component of helicidum or another glyco-benzaldehyde is substituted by another sugar form amenable to uptake by mammalian cells in anaerobiosis (e.g., anaerobic tumor cells or virally-infected cells).

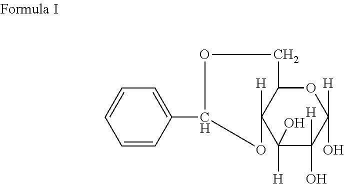

Salicinium® has unique and previously unreported mechanisms of action. In exemplary case studies disclosed herein, qualified clinicians administered Salicinium according to protocols described below to diverse Stage IV cancer patients. The subject treatments extended patient survival to an extraordinary and surprising degree. As of the date of this disclosure, physicians and naturopaths working with the inventors have administered Salicinium clinically to a total of 675 patients, over a continuous study period of more than 10 years, and from this work Salicinium has been clinically proven to exert potent, therapeutic anti-cancer effects, including to extend five-year survival rates of Stage IV cancer patients well beyond median survival rates for all groups studied.

In additional studies herein, Salicinium®, exemplified by the glyco-benzaldehyde helicidum, is demonstrated to have potent immune-enhancing effects. In one embodiment, Salicinium mediates powerful immune stimulatory effects by modulating alpha-N-acetylgalactosaminidase (nagalase) physiology and circulating nagalase blood levels in patients with cancer or viral infections. In addition, Salicinium exerts immune modulatory therapeutic benefits by modulating the activity, proliferation and/or anti-cancer or anti-viral activity of one or more immune effector cells (e.g., macrophages, T cells, natural killer (NK) cells and B cells).

Much of the evidence supporting the effectiveness of Salicinium® is derived from its extensive continuing use in clinical practice based on the pioneering studies described here. In addition, the evidence provided herein includes in vitro, cellular and other pre-clinical studies that diverse labs have performed under the direction of the inventors—all of which validate the novel and potent anti-cancer and immune-modulatory effects of Salicinium and elucidate its previously unknown mechanisms of action.

Salicinium® includes a variety of candidate glyco-benzaldehydes that will be proven effective within the methods and compositions of the invention. This efficacy is illustrated in examples below using the simplest, naturally occurring glyco-benzaldehyde helicidum (or helicin). Helicidum was initially identified as a plant-derived glyco-benzaldehyde, originally helicidum was extracted from Helicia essatia (Hook), Helicia nilgrinica (Bedd), or Helicia hilagirica (Bedd), all plants indigenous to Western China. Presently helicidum is available in the US and elsewhere from multiple companies that produce synthetic versions of the compound. Either naturally-derived or synthetic versions of helicidum are useful within the Salicinium® compositions and methods of the invention. Typically, the purity of the helicidum starting material will be at least 70-90% w/w, and in most cases the purity will be above 90%, 95% or even 97-98% w/w.

Helicidum (CAS No. 80154-34-3) has alternative chemical names, including 4-(beta-D-allopyranosyloxy)-benzaldehyde, 4, 6-0-benzylidine-D-glucopyranosyloxy, and 4-formylphenyl-0-β-D-allopyranoside, and formaldehydephenlyl-O-Beta-d-pyranosyl alloside, with the following standard structure and molecular particulars:

### Helicidum Molecular Structure and Particulars

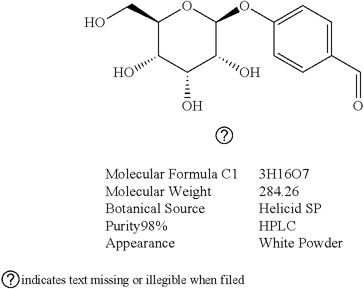 [SMILES: O=Cc1ccc(O[C@@H]2O[C@H](CO)[C@@H](O)[C@@H](O)[C@H]2O)cc1]

In addition to natural or synthetic helicidum, other known, naturally-occurring or synthetic, glyco-benzaldehydes can be routinely selected as candidates for use within the invention, and routinely tested according to the teachings herein to determine operability within the claimed anti-cancer, anti-viral and other treatment methods and pharmaceutical compositions of the invention.

Although there has been a diverse array of benzaldehydes, and some glyco-benzaldehydes, proposed for a wide variety of therapeutic uses, the disclosure herein represents the first demonstration of effective treatment of cancer and viral infections using a glyco-benzaldehyde. While dozens of purported therapeutic uses have been speculatively proposed for helicidum and other glyco-benzaldehydes (e.g., as traditional botanical remedies), no scientific clinical utility has ever been demonstrated or approved for these compounds in the treatment of cancer, viral infection, or other contemplated clinical uses. With respect to cancer and viral infection, no substantial clinical testing and proof of therapeutic efficacy in clinical applications has ever been validated for these interesting compounds.

Within more detailed embodiments of the invention, Salicinium® comprises helicidum or a functional analog or derivative of helicidum demonstrably effective within the anti-cancer or anti-viral methods of the invention. Helicidum analogs and derivatives may have any functional group of the core molecule altered, for example by chemical substitution, to seek improvements in one or more biological properties of the active compound (e.g., solubility, bioavailability, permeation, transport, half-life, etc.) Exemplary studies to identify new drug candidates from rational design chemical derivation of helicidum are provided by Wei et al. (Bioorganic & Medicinal Chemistry Letters, 18(24) pp 6490-6493 (2008), which identified an interesting helicidum analogue (Formula II below) bearing a 4,6-O-benzylidene substituent on the sugar moiety.

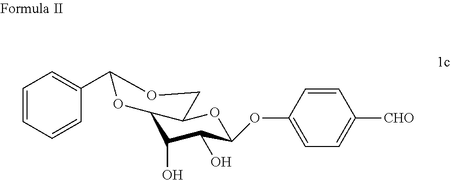

This and other rationally-designed synthetic analogues and derivatives of helicidum will be useful to select and evaluate operable new drug candidates for use within the Salicinium® compositions and methods herein.

In yet additional embodiments of the invention a Salicinium® composition for pharmaceutical use comprises one or more benzaldehyde derivatives including, but not limited to, those represented by Formulas III-V, below, intermediaries of Formulas III-V, and precursors and metabolites to these benzaldehyde compounds (see, e.g., Formulas VI-VII).

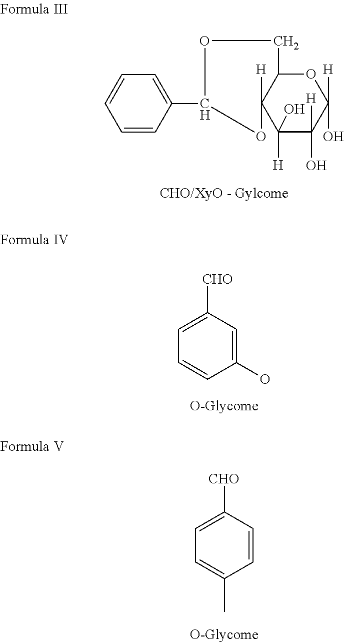

Relating to the above Formulae, the glycome (represented in Formula I by glucose) can be any carbohydrate or sugar including, but not limited to, any one of the hexoses including, but not limited to, the a or f3 forms of glucose, mannose, galactose, fructose, or a biose formed from any two of the above, wherein the two hexoses may be the same or different.

Exemplary glyco-benzaldehyde alternative compounds for use within the Salicinium® formulations and methods of the invention include, but are not limited to, 4, 6-0-benzylidine-D-glucopyranosyloxy, 2-β-D-glucopyranosyloxy benzaldehyde, 3-β-D-glucopyranosyloxy benzaldehyde, and 4-β-D-glucopyranosyloxy benzaldehyde. Additional useful forms and derivatives of Salicinium for use within the invention include other pharmaceutically acceptable active salts of these exemplary benzaldehyde compounds, as well as active isomers, enantiomers, polymorphs, intermediaries, precursors, solvates, hydrates, and/or prodrugs of said compounds. In representative examples, useful precursors and intermediaries of 4, 6-0-benzylidine-D-glucopyranosyloxy, 2-β-D-glucopyranosyloxy benzaldehyde, 3-β-D-glucopyranosyloxy benzaldehyde, and 4-β-D-glucopyranosyloxy benzaldehyde can be routinely designed, selected and tested for use within the therapeutic methods and compositions of the invention, including without limitation 2(hydroxymethyl) phenyl-β-D-glucopyranoside as seen in Formula VI, below, 3-(hydroxymethyl)phenyl-β-D-glucopyranoside as seen in Formula VII, below, or 4-(hydroxymethyl)phenyl-β-D-glucopyranoside; and intermediate compounds such as, but not limited to, 2-hydroxybenzaldehyde, 3-hydroxybenzaldehyde, and 4-hydroxybenzaldehyde which convert to salicylic acid, 3-hydroxysalicylic acid, and 4-hydroxysalicylic acid respectively, or any other pharmaceutically acceptable active salts of said compounds, as well as active isomers, enantiomers, polymorphs, intermediaries, precursors, solvates, hydrates, and/or prodrugs of said compounds.

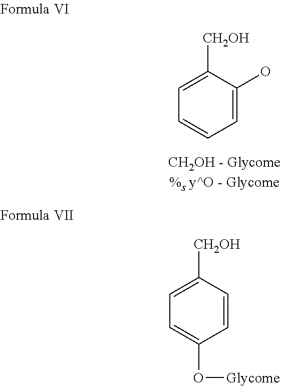

Likewise, in the foregoing Formulae VI and VII the glycome may be any carbohydrate or sugar including, but not limited to any form of the hexoses, including the a and f3 forms of glucose, mannose, galactose, and fructose, or a biose formed from any two of the hexoses, wherein the hexoses may be the same or different.

In exemplary embodiments, the Salicinium® compositions and methods of the invention employ a compound, analog or derivative of Formula I-V, a precursor compound of Formula VI-VII, or an intermediary compound of these or another glyco-benzaldehyde, alone or in combination, within an anti-cancer, anti-viral or immune modulatory Salicinium composition.

In related embodiments, therapeutic methods are provided that employ a Salicinium® composition to treat and/or prevent symptoms of a cellular proliferative disorder, for example cancer, or another disease or condition associated with cancer.

In other aspects of the invention, Salicinium® is administered as an anti-viral or anti-microbial effective agent within therapeutic methods and compositions.

Mammalian subjects amenable to treatment with Salicinium® (including benzaldehyde derivatives, glyco-benzaldehydes and related compounds according to the teachings herein) include, but are not limited to, human and veterinarian subjects suffering from cellular proliferative disorders generally (e.g., hyperplasia of various tissues and organs, endometriosis, psoriasis), and in more specific embodiments cancer (in all of its stages, primary and secondary tissue targets, and proliferative forms).

Exemplary forms of cancer amenable to treatment using Salicinium® and related compositions of the invention include, but are not limited to, breast cancer, lung cancer, prostate cancer, skin cancer including melanoma, liver cancer, thyroid cancer, esophageal cancer, sarcoma, brain cancer of all types, colon and rectal cancers, bladder cancer, gall bladder cancer, stomach cancer, renal cancer, ovarian cancer, uterine cancer, cervical cancer, non-Hodgkin's lymphoma, acute myelogenous leukemia (AML), acute lymphocytic leukemia, chronic lymphocytic leukemia (CLL), myeloma, mesothelioma, pancreatic cancer, Hodgkin's disease, testicular cancer, Waldenstrom's disease, head/neck cancer, cancer of the tongue, and malignancies induced by SV40 virus. Subjects amenable to treatment may have cellular proliferative disorders at any stage of development including, but not limited to, challenging stage III and stage IV forms of cancer.

In exemplary embodiments, the Salicinium compounds, formulations and methods of the invention substantially prolong mean survival of mammalian stage III or stage IV cancer patients, including veterinary patients and humans. In more detailed embodiments documented herein, Salicinium methods and compositions of the invention substantially extend survival in stage IV human cancer patients.

In certain aspects of the invention, Salicinium® (e.g., comprising a helicidum glyco-benzaldehyde) is administered according to a novel, intravenous delivery protocol, optionally followed by an oral delivery/treatment protocol. These novel protocols profoundly extend survival among stage IV human cancer patients, even among study subjects who exhibit resistant or intractable forms of cancer (i.e., who present after one or more aggressive rounds of conventional oncotherapy, such as chemotherapy, radiation, surgery and/or hormonal therapy), with active and unstable metastatic disease, or who otherwise are not fit for, or who do not respond to, conventional cancer treatments such as chemotherapy.

More generally, subjects amenable to treatment employing Salicinium® methods and compositions of the invention may include any mammalian subject suffering from a disease that results in cells or tissues (e.g., cancer cells or virally-infected cells), exhibiting metabolic conversion to an obligately anaerobic state (anaerobiosis). Salicinium® compounds effectively target and are actively transported into anaerobic cells, and once taken up into cells the active compounds disrupt glycolytic and synthetic mechanisms in the cells. Among the most surprising discoveries presented here, Salicinium® not only disrupts glycolysis and normal cellular synthetic processes, it potently induces apoptosis in cancer cells and other obligate anaerobic cells.

The instant description demonstrates the surprising potency of Salicinium® formulations (e.g., comprising helicidum or another useful glyco-benzaldehyde), as novel therapeutic tools for treating cancer and other afflictions characterized by anaerobiosis. In more detailed aspects, Salicinium is administered to induce apoptosis in anaerobic cancer and/or viral-infected cells (including cells infected by cancer-causing “oncoviruses”, such as HPV. In other detailed embodiments, Salicinium targets cells with upregulated sugar receptors and induces apoptosis in these cells. In yet additional embodiments, Salicinium is administered to cancer or viral-infected patients exhibiting increased plasma levels of nagalase, wherein Salicinium either induces apoptosis in anaerobic cells expressing high levels of nagalase (in the case of cancer cells) and/or disrupts nagalase expression in viral-infected cells. In related embodiments, Salicinium treatment induces or enhances one or more immune responses (e.g., activates or induces macrophages, T cells, natural killer (NK) cells and/or B cells). In certain embodiments Salicinium powerfully down-regulates the blood and tissue levels, and activity, of nagalase, and thereby relieves nagalase-mediated immune suppression (see below). Through this mechanism Salicinium® can be administered to elicit a potent cellular response against cancer or viral-infected cells, in addition to Salicinium's direct anti-cancer and anti-viral efficacy.

Many studies have been undertaken in the course of the inventors' work to demonstrate that Salicinium® (e.g., helicidum or another glyco-benzaldehyde) is rapidly and efficiently taken up by anaerobic mammalian cells, including cancer cells and other proliferative or pathogen-challenged cells in vitro and in vivo. This active uptake mechanism allows for targeted delivery and loading of Salicinium as an effective therapeutic agent directly into targeted (anaerobic) cells in need of anti-cancer or anti-viral therapy. This targeted delivery of Salicinium directly into anaerobic cells mediates apoptosis and disruption of nagalase production in cancer cells, and ablates nagalase production in viral-infected cells, in both cases mediating a subsequent boost of the immune system in treated subjects.

Mammalian cells obtain oxygen through the process of respiration, which takes place in the mitochondria. Through respiration the mitochondria produce ATP (adenosine triphosphate).

Dr. Otto Warburg discovered that by lowering oxygen levels of normal cells by 35%, they can continue to live without respiration. All mammalian cells can use this anaerobic (“without oxygen”) process to help them survive short periods of stress. However, should a cell suffer longer-term stress, it metabolically converts to “anaerobiosis”. It was long presumed that simply adding oxygen back to fermenting cells (e.g., through the use of antioxidants such as high-dose vitamin C, or hyperbaric oxygen therapy) would convert these cells back to healthy aerobic respiration. This does not in fact occur. Rather, once a cell's metabolism shifts to anaerobic “fermentation”, this becomes an “obligate” metabolic state—meaning the cell continues anaerobic fermentation even when oxygen is restored to its environment.

Anaerobic fermenting cells are predisposed to de-differentiate and become cancerous. Fermenting cells generate only about 5% as much ATP as normal respiring cells. Instead of utilizing oxygen in ordinary respiration they ferment simple sugars. When compared to normal cells, fermenting cells have many more sugar receptors. This is part of the mechanism whereby anaerobic cancer and viral-infected cells secure sufficient energy to proliferate rapidly and manufacture more cells and viral replicants.

Glycolysis is the process by which the body produces ATP along with NAD and NADH. The enzyme HK is the first enzyme in the glycolysis pathway. When Salicinium® enters an anaerobic cancer or viral-infected cell, the cytoplasm of the cell has no free ATP to recycle back into the glycolytic cycle to produce pyruvate (essential for normal cellular respiration). In the presence of a glyco-benzaldehyde Salicinium compound, this process is altered. Once inside the anaerobic cell, Salicinium disrupts NAD, NADH, and ATP development. Further mechanistic studies developed here clarify this activity of Salicinium, particularly the disruption of NAD/NADH, which serves to potently induce apoptosis in anaerobic cells, including virtually all cancers and a variety of viral-infected cells.

Salicinium® in the form of a glycome (sugar) conjugated (attached) to a benzaldehyde or other toxic moiety is capable of disrupting the glycolytic pathway upon entry into a fermenting cell. In the case of one exemplary Salicinium® compound helicidum, benzaldehyde is attached to a sugar molecule and is thus readily accepted into anaerobic cells through the glucose transporter (GLUT) pathway. GLUTs are present in all cell types, but cancer cells typically overexpress GLUTs. In the GLUT transportation pathway, benzaldehyde is met immediately by the enzyme hexokinase II (HK2) and through enzymatic reaction with ATP is changed to glucose 6-phosphate-benzaldehyde (G 6-p-b). G 6-p-b, again through a further enzymatic reaction and another investment of ATP, becomes fructose 1 6-bisphosphate-benzaldehyde (FBP-b). Most of the glucose and fructose that provide energy to anaerobic cells are converted into FBP.

Salicinium® is effective in the compositions and methods of the invention by virtue that it irreversibly modifies the activities of HK2, G 6-P, F 6-p, and FBP, in part by altering their chemical structure, electrical potential and/or substrate recognition/binding/interaction potentials. In more detailed mechanistic aspects, Salicinium® alters the physiology of a key metabolic enzyme pyruvate kinase (PK). Most tissues express either PK1 or PK2. PK1 is found in normal differentiated tissues, whereas PK2 is expressed in most proliferating cells, including all cancer cell lines and tumors tested to date. Although PK1 and PK2 are highly similar in amino acid sequence they have different catalytic and regulatory properties. PK1 has high constitutive enzymatic activity. In contrast, PK2 is much less active but is allosterically activated by the upstream glycolytic metabolite fructose 1, 6-bisphosphate (FBP). PK enzymes are generally inhibited by ATP, and in the case of the downstream PK2 enzyme its activity is held in check by ATP until FBP activates it. Relevant here, Salicinium® (exemplified by a glycome bound with a benzaldehyde or other glycolysis-disruptive moiety) is converted into an unnatural FBP-b analog. In this state Salicinium disrupts the HK2 pathway. With no upstream glycolytic metabolite having interactive potential with the low energy PK2 enzyme, normal FBP metabolism is irreversibly changed, and when HK2 enzymes interact with Salicinium upon its entry through the GLUT pore PK2 activity is likewise halted.

Salicinium® also interacts adversely with nicotinamide adenine dinucleotide phosphate (NADP). NADP plays an important role in the oxidation-reduction involved in protecting against toxicity of reactive oxygen species (ROS). Salicinium's interaction with HK-II upon entry into anaerobic cells irreversibly disrupts NADP metabolism, whereby the glycolytic energy function of the anaerobic cell becomes completely dysfunctional, with the result being induction of apoptosis.

In more detailed mechanistic aspects, Salicinium® interacts in the glycolytic pathway when anaerobiosis triggers conversion of pyruvate to lactic acid by fermentation. During lactic acid fermentation, pyruvate and NADH are converted to lactic acid and NAD+. NAD+ is also used in glycolysis to generate ATP in which C6Hi2O6+2ATP+2NAD+=>2pyruvate+4ATP+2NADH. Cancer cells create a slightly acidic intracellular environment (cancer cell pH is about 7.00, whereas normal cellular pH is about 7.36) contributing to metabolic conversion of normal, aerobic cells into fermenting cells. In the process of fermentation, Salicinium (e.g., a glyco-benzaldehyde such as helicidum) functions as a deactivator of NAD+. Upon entry into the cytosol, benzaldehyde (and other comparable effectors linked to a carrier glycome for targeted cellular delivery) reduces NAD+ to NADH+H, blocking the normal function of NAD+, interfering with the normal acid detoxification process, and resulting in a decrease in pH (due to the inability to convert pyruvic acid to lactic acid)—powerfully disrupting glycolysis in fermenting cells, stopping unregulated growth of fermenting (e.g., cancer) cells, and ultimately inducing cellular apoptosis.

These and other discoveries herein provide for novel compositions and methods to disrupt cellular metabolism, biosynthetic function, nagalase expression, and viability of anaerobic cancer cells and viral-infected cells.

The role of Salicinium® in disrupting cellular metabolism and biosynthesis in anaerobic cancer cells elucidates yet another potent activity of Salicinium, namely an ability to powerfully disrupt biosynthesis in other aberrant proliferative disease conditions, and in viral-infected cells (even when those cells may not be metabolically converted to obligate anaerobic metabolism). In the case of non-cancerous proliferative cells and most cells infected with active viruses (i.e., non-dormant viruses that actively commandeer host cell biosynthetic machinery to replicate new virions), the diseased or host cell expresses elevated levels of GLUT receptors and otherwise upregulates passive and active uptake of sugars (including Salicinium “sugar-toxin” compounds, exemplified by glyco-benzaldehydes) into the diseased or viral host cell. Data determined herein reveal that GLUT-mediated and other glycome transport mechanisms routinely increase Salicinium uptake into proliferating cancer and non-cancer cells and viral-infected cells by 50-100%, often 2-5 times, up to 10-15 times greater, or higher compared to normal receptor levels and sugar transport rates in healthy cells.

With this common upregulation of GLUT and other cellular transporters of sugars and related molecules, including Salicinium®, diseased proliferative and viral-infected cells are vulnerable to Salicinium-mediated disruption of biosynthesis in these cells, via the same disruption of glycolytic pathway targets described in detail above. A critical result discovered herein relates to Salicinium's potent down-regulation of a specific biosynthetic product, alpha-N-acetylgalactosaminidase (nagalase) which is critical for immunes suppression and immune evasion by cancer and other proliferative cells, and viral-infected cells.

Nagalase is a protein made by all cancer cells and a wide diversity of viruses (including human immunodeficiency virus (HIV), hepatitis B, hepatitis C, influenza, herpes viruses, human T lymphotropic virus (HTLV), Epstein-Barr virus, cytomegalovirus (CMV) and others). A growing body of evidence indicates that nagalase potently suppresses immune functions and frequently causes immunodeficiency in cancer patients and patients carrying a pathogenic viral load.

Nagalase impairs immune function as an immune masking protein that is overexpressed on the surface of cancer cells and integrated envelopes of many viruses. A particularly harmful role of nagalase in mediating immune-suppression is to block production and/or impair normal activity of Gc protein-derived Macrophage Activating Factor (GcMAF). Nagalase functions as an enzyme that inactivates GcMAF and its natural precursor Gc-globulin (or Gc protein, also known as vitamin D binding protein (VDBP)). In this manner nagalase blocks or impairs normal function of GcMAF in regulating critical immune responses mediated by macrophages. In more detailed terms, serum VDBP is the precursor for GcMAF, and nagalase deglycosylates (removes sugars from) DBP, short-circuiting GcMAF production resulting in immunosuppression. (see, e.g., Yamamoto et al., Pathogenic Significance of Alpha-N-Acetylgalactosaminidase Activity Found in the Hemagglutinin of Influenza Virus. Microbes Infect. 7(4):674-81 April 2005).

Globulin component Macrophage Activating Factor or GcMAF is also called vitamin D-binding protein-derived macrophage activating factor. GcMAF is critical to normal immune system function, and is notably depleted in individuals with suppressed immune system function (most commonly in patients with cancer and viral infections associated with abnormally high levels of nagalase. GcMAF is a critical signaling factor involved in the activation and programming of key immune effector cells known as macrophages. Reduction or impairment of GcMAF disrupts normal macrophage function and results in profound downstream immune dysfunction.

Macrophages originate in the bone marrow where they differentiate through the myeloid lineage through monoblast and promonocyte stages to the monocyte stage, then monocytes enter the blood and tissues where they mature into macrophages. Macrophages are large and usually immobile, but become motile when stimulated by inflammatory cytokines. Macrophage functions include phagocytosis and pinocytosis, presentation of antigens to T and B lymphocytes, and secretion of a variety of products, including cytokines, enzymes, complement components, coagulation factors, prostaglandins and leukotrienes, and several other immune regulatory molecules. In this context macrophages can be viewed as a central hub in the immune system, mediating several immune signaling and activation cascades. Disrupting macrophage function and activity thereby disables a multitude of primary immune functions, including T and B cell activation and programming, thereby fundamentally disrupting cell-mediated and humoral immunity.

Macrophages themselves are primary effector cells in the immune system, and when activated they become motile and seek out cancer and viral-infected cells (at least those that are not protectively “masked” against immune-surveillance by nagalase), engulfing those diseased cells by phagocytosis, and eliminating cellular debris (e.g., debris resulting from cancer apoptosis, or immune destruction of viral-infected cells), free microbes, virions, and foreign proteins. Apart from this primary phagocytosis role they play a critical role in nonspecific defense and also help initiate specific defense mechanisms by recruiting other immune cells such as lymphocytes. For example, they are important as antigen presenters to T cells. Macrophages have several different receptors on their surface that help them effectively identify and bind pathogens to promote phagocytosis and stimulate the release of cytokines. These receptors include: IL-1, IL-6, CXCL8, IL-12, and TNF-α Inflammatory cytokines released by the macrophages have the ability to stimulate effects at a site of the infection (local) and throughout the body (systemic). Among these effects mediated by macrophage inflammatory cytokines, the recruitment of neutrophils to sites of infection by CXCL8, and activation of NK cells by IL-12, are critically dependent on macrophage function.

Natural killer (NK) cells are a part of the lymphoid linage of white blood cells. They are large granular lymphocytes that represent about 10-15% of circulating lymphocytes in the blood. Cytokines secreted from macrophages activate and facilitate the entry of NK cells into tissues to attack cancer cells and eliminate viral and other infected or stressed cells, through various pathways, including the normal cell-killing function and release of cytokines triggering downstream anti-cancer and anti-viral immune responses.

To control viral infections the body's immune system normally responds to viruses by secreting cytokines, which normally function to disrupt viral replication and make cells more susceptible to attack by NK cells. The primary cytokine released by NK cells is type II interferon, which activates macrophages. The macrophage secretion of IL-12 and the NK cell secretion of type II interferon create positive feedback signals that cooperatively increase activation of both types of cells (macrophages and NK cells) within infected tissues. These processes enhance macrophage and NK cellular activity and prevent infections from spreading. In addition, the activation of macrophages by type II interferon leads to release of cytokines that also aid in the activation of T cells. Activation of T cells jumpstarts the adaptive immune response and allows cytotoxic T cells to take over after NK cell responses are complete.

In view of the foregoing, the invention provides powerful new tools and methods that employ Salicinium® to sharply reduce immunosuppressive levels of nagalase in patients with cancer and other proliferative disorders. In related methods, Salicinium is administered in an effective amount and delivery method to relieve immunosuppression in subjects with chronic or high load viral infections. According to the teachings herein, Salicinium (e.g., a glyco-benzaldehyde compound) is administered in an anti-nagalase effective amount to the subject, sufficient to disrupt nagalase synthesis, lower nagalase levels (e.g., in a tumor or cancerous tissue, on cell surfaces of cancer or viral-infected cells, in an envelope of circulating viral particles, or in circulating plasma or other compartments within the subject).

Related clinical management and treatment compositions and methods of the invention typically involve first diagnosing a patient with advanced, e.g., Stage III or Stage IV, cancer, or a chronic viral infection with high titer of virus detected (e.g., as determined by conventional viral detection/quantification ELISA assays measuring viral proteins or antiviral antibodies, viral RNA or viral DNA detection/quantification methods using polymerase-based assays) in a patient blood sample. Subjects testing positive for seriously elevated nagalase (e.g., above 0.90 Units, or 0.90 nmol/min/mg) will be selected for either intravenous or oral Salicinium treatment as described herein. Treatment intensity and mode will depend on other patient factors, including other indicia of morbidity of cancer symptoms or viral infection sequelae (e.g., diagnostic data relating to tumor load, CTC levels, circulating blood markers, etc. for cancer subjects, general health indicators and patient self-reporting for EBV patients, T Cell population levels for HIV patients, hepatic functional markers for hepatitis C subjects, etc.)

Some patients with the highest tumor or viral loads and severe symptomology (determined here to correspond to high levels of immunosuppressive nagalase, for example in the range of 1.5-2.5 Units) will be treated more aggressively, for example using the aggressive iv Salicinium protocol described in Example II. Other patients may be treated and managed effectively for lower severity cancer disease, or lower viral load and less severe symptomatic viral infections, using an abbreviated iv Salicinium treatment protocol (e.g., 5-10 iv infusions over 1-2 weeks), or even exclusively an oral Salicinium treatment method.

Anti-nagalase, anti-cancer and anti-viral treatment methods aimed at modulating nagalase and relieving immunosuppression will further typically involve following up on patient status by multiple nagalase blood tests over time to monitor patient response to therapy. In working examples presented herein, subjects treated with Salicinium were monitored for nagalase reduction, coordinately with monitoring of cancer symptom abatement, and/or viral clearance over time, for a study course of many months to a year, with staged monitoring at, e.g., one month, two months, three months, six months and 12 month milestones post-initiation of treatment. Based on comparable staged monitoring assays, treatment and clinical management methods of the invention further contemplate altering an intensity, modality or dosing regimen of Salicinium treatment for a patient based on observed correlated changes in one or more diagnostic indicia selected from 1) pre- and/or post-treatment blood nagalase levels; 2) pre- and/or post-treatment cancer diagnostic indicia observed (e.g., quantified tumor load, CTCs, blood markers, etc.) and/or 3) pre- and/or post-treatment observation of viral titer/load and/or patient symptoms of viral infection incidence, severity, abatement or progression.

In related aspects, clinical management and therapeutic efficacy of Salicinium treatments can be further modified or optimized by coordinate monitoring of immune function indicators, for example by detecting pre- and/or post-treatment numbers of immune effector cells (e.g., macrophages, NK cells, T cytotoxic cells or B cells), quantifying activation indicators of immune effector cells (e.g., detecting cytokine expression or NK cellular signaling/activation by macrophages, measuring NK killing activity against tumor cells or viral-infected cells (as in the working examples presented below), measuring circulating antibodies produce by macrophage-activated B cells, or directly measuring effector immune cell destruction of cancer cells, viral-infected cells, or circulating free virions in treated patients (e.g., by in vivo visualization techniques, blood assays, biopsy, and other methods).

Within the foregoing methods nagalase levels can be routinely monitored to assess efficacy of different Salicinium® compounds, to determine initial and subsequent treatment protocols, to monitor and clinically manage, patients undergoing Salicinium treatment, and to validate clinical endpoints for patients cleared of cancer and/or viral infection through the course of Salicinium treatment. At the core of these methods is the use of Salicinium to mediate reduction of nagalase overexpression and alleviate nagalase-mediated immunosuppression. A corollary benefit of these methods is that Salicinium indirectly mediates activation of powerful immune responses, including cellular and humoral immune responses that target and effectuate inactivation, cell-killing, phagocytosis and debris clearing (including in the case of apoptotic cancer cells) of cancer cells and viral infected cells.

In certain embodiments, clinical management tools and methods of the invention begin patient evaluation and treatment by testing for abnormally high, immunosuppressive levels or activity of α-N-acetylgalactosaminidase (nagalase) in a serum or plasma of the patient, as may be determined by any of a variety of well-known assay methods. These assays are typically integrated in a clinical management series of nagalase tests staged throughout a course of treatment and monitoring of cancer and viral-diseased patients. Some conventional nagalase assays that may be used measure simple blood levels of nagalase, whereas other testing methods quantify nagalase by detecting its enzymatic activity (in one role nagalase functions as an extracellular matrix-degrading enzyme secreted by cancerous cells to facilitate the process of tumor invasion, whereas nagalase also appears as intrinsic component of viral envelope proteins, as shown for HIV, influenza and many other viruses. Various commercial laboratories provide nagalase testing services and kits, including Redlabs in Belgium, and ELN in Holland. These and other assay methods are widely known and published. The testing used herein follows general protocols presented in Yamamoto et al., British Journal of Cancer: 77(6), 1009-1014, (1998), Reddi et al., Cancer Lett 29; 158(1):61-4 (2000), Yamamoto et al., Microbes Infect. 7(4):674-81 (2005); and Yamamoto, AIDS Res Hum Retroviruses 22(3):262-71 (2006), each incorporated herein by reference.

For nagalase testing on plasma whole blood must is collected in EDTA-coated tubes, and these are centrifuged within one hour of collection at 3,000 rpm for 10 minutes.

Plasma is then aliquoted into new, sterile tubes and frozen for subsequent shipping to the testing lab on dry ice. For nagalase testing on serum whole blood is collected in serum tubes (e.g., BD Vacutainer tubes), which are likewise centrifuged, aliquoted and frozen for shipping.

Nagalase activity in serum or plasma is measured kinetically through conversion of a fluorogenic substrate in function of time. The test is standardized against data developed from a pool of healthy persons (normal WBC count, no inflammation, CRP<1 mg/L, no clinical history of immune disease or diabetes), which has established a conventional “normal” range of nagalase of from 0.5 to 0.95 Units (nMol/ml/min) for adults (though we regard values above 0.65 Units as abnormal, or at least suspect).

In a first assay step nagalase is captured on a solid phase coated with a α-N-acetylgalactosaminidase-specific antibody able to capture up to 10 ng/ml of nagalase from serum or plasma. After removal of unbound material, the activity of immobilized Nagalase is measured by incubation with a specific α-N-acetylgalactosaminidase fluorogenic substrate. The resulting nagalase enzymatic activity is expressed as nmol/min per milliliter.

Nagalase testing according to the methods of the invention will also frequently provide for detection of undiagnosed cancers and/or viral infections. In this context, methods for prophylactic or early treatment of cancer and viral infection are contemplated, wherein subject presenting with a suspect nagalase level of greater than 65 Units, or greater than 95 Units, will be further evaluated for prospective cancer or viral infection (using standard cancer and viral diagnostic test methods), and Salicinium® treatment can be initiated without prior conventional detection (i.e., prior to the nagalase screen results are determined). This enables earlier, less costly and less invasive treatment options. Whereas nagalase is normally expressed in only trace levels in the blood of healthy subjects, it appears elevated in the blood stream when only nascent cancer or viral infections are present. Because nagalase may be elevated by just a small group of abnormal cells, which may in fact be “pre-cancerous”, Salicinium® treatment will in some embodiments be initiated as an effective prophylactic measure, based exclusively on an elevated nagalase report (e.g., in subjects with other cancer or viral infection risk factors, such as a family history of cancer, a viral-infected intimate partner, etc.)

Rising nagalase levels indicate a cancer or virus is growing and spreading in the subject, and more aggressive Salicinium® treatment methods will thereafter be employed. Conversely, nagalase levels will decrease as cancer or infection is cleared through the various activities of Salicinium (namely: 1) Inducing cancer apoptosis; 2) Disrupting or ablating nagalase expression by cancer and viral-infected cells; 3) Unmasking cancer and viral-infected cells by stripping their nagalase surface coating, allowing for immunosurveillance and active immune targeting of these unmasked cells; 4) Reversing immunosuppression mediated by nagalase acting to inhibit GcMAF conversion; and 5) indirectly activating various immune effector systems through nagalase clearance, resulting dis-inhibition of GcMAF, attendant potentiation of GcMAF activation of macrophages and downstream macrophage immune-stimulatory activities (including cytokine-mediated induction/activation of T cells and NK cells).

Non-Salicinium treatment methods may incidentally lower nagalase levels and achieve certain comparable therapeutic benefits as described above. For example, many unrelated treatments that reduce cancer or viral load (e.g., surgery, chemotherapy, radiation, antiretroviral treatment, etc.) will typically also lower nagalase levels. In this regard, patient treatment history and current status will be integrated into nagalase-assay-based clinical monitoring and management protocols of the invention.

In many patients, both cancer and viral infection occur simultaneously, and the examples herein below show actual clinical data that such co-morbid conditions can be effectively treated with Salicinium®. Oncogenic viruses represent one target of these coordinate treatment methods, resulting in a multitude of malignancies that are directly caused by chronic or spontaneous viral infection (causing infected host cell “transformation” to a cancerous phenotype). Although many of the molecular signaling pathways that underlie virus-mediated cellular transformation are known, the impact of these viruses on metabolic signaling and phenotype within proliferating tumor cells is less well understood. Like cancer cells, cells infected with oncogenic viruses metabolically transform to a phenotype characterized by glucose uptake and obligate glycolysis, with dysregulation of molecular pathways that regulate oxidative stress. Through their effects on cell proliferation pathways, such as the PI3K and MAPK pathways, the cell cycle regulatory proteins p53 and ATM, and the cell stress response proteins HIF-1α and AMPK, viruses exert control over critical metabolic signaling cascades. In one critical aspect, oncogenic viruses modulate tumor metabolism by direct and indirect interactions with glucose transporters, such as GLUT1, and specific glycolytic enzymes, including pyruvate kinase, glucose 6-phosphate dehydrogenase, and hexokinase. Through these pathways oncogenic viruses alter the energy-use machinery of transformed cells, making them amenable to the same Salicinium-mediated glycolytic disruption and attendant apoptosis described above for ordinary cancer cells.

The anti-nagalase and immune-stimulatory methods herein are directed toward inducing or enhancing an immune response in a mammalian subject suffering from cancer or viral infection, by reducing or eliminating a blood level of alpha-N-acetylgalactosaminidase (nagalase) in the subject. Treatment methods involve administering an effective amount of a composition comprising a nagalase-reducing Salicinium® (e.g., a glyco-benzaldehyde) compound, effective to reduce or ablate synthesis of nagalase in cancer cells and virus-infected cells, whereby circulating plasma levels of nagalase in the subject are reduced or eliminated and nagalase suppression of immune function in the subject is alleviated. Subsequently the immune response in the subject is initiated or enhanced by disruption and removal of nagalase as an immune-suppressive factor, typically involving a release or reversal of GcMAF suppression by nagalase.

In related methods, Salicinium® serves a critical anti-cancer and immune-enhancing role by causing unhealthy cells to release their nagalase coating, thus allowing the immune system to recognized “unmasked” cancer and viral-infected cells, and re-activate against these cells (e.g., by activation of macrophages and natural killer (NK) cells that directly target and attack cancer and viral infected cells, no longer protected by high nagalase levels on their surface). This surprising anti-cancer and immune-modulatory activity has been documented in various laboratory studies herein, demonstrating Salicinium-mediated, therapeutic changes in nagalase levels in patients, and attendant activation or enhancement of the immune system (e.g., activation of immune effector cells such as macrophages and NK cells). After treatment with Salicinium® nagalase levels drop in treated cancer and viral-infected patients (and in their immune cell cultures), while concurrently immune system functions re-activate or increase (e.g., NK cell populations rise, NK migration is activated, NK cell cancer-killing activity is greatly enhanced, other white blood cell counts and immunoglobulin levels rise, etc.)

In addition to oncoviruses and other chronic viral infections, individuals suffering from cancer and other cellular proliferative disorders frequently exhibit other secondary infections, for example microbial infections such as bacterial and fungal infections, including but not limited to Lyme disease, candidiasis, and methicillin resistant Staphylococcus infections. Combinatorial and coordinate treatment protocols of the present invention may be used to treat such secondary infections using, for example, anti-microbials which may be used in combination with a benzaldehyde derivative compound of Formula I-V, or precursor or intermediate compound of Formula V or VI.

In one illustrative embodiment of the invention, Salicinium® compositions are employed to treat human papilloma viruses (HPVs). HPVs are a group of more than 150 related viruses that cause papillomas, more commonly known as warts. Some types of HPV only grow in skin, while others grow in mucous membranes such as the mouth, throat, or vagina. All types of HPV are spread by contact, and more than 40 types of HPV can be passed on through sexual contact. Most sexually active people are infected with one or more of these HPV types at some point in their lives. At least a dozen of these types is known to cause cancer. By virtue of their high frequency and oncogenic activity, HPVs are particularly well-suited to treatment using Salicinium, with its multiple activities and attack modes.

There are no effective medicines or other treatments for HPV, apart from removing or destroying cells known to be infected. In most people, the body's immune system controls HPV infection and gets rid of it over time, but this is not the case with cancer or viral infected subjects that are immunosuppressed. As noted, certain HPV strains are implicated as the main cause of cervical cancer, the second most common cancer among women worldwide. Cervical cancer has become much less common in the United States, after the Pap became widely available for many years. This test can reveal pre-cancerous changes in cells of the cervix that might be caused by HPV infection, and the abnormal tissue can be destroyed or removed before it progresses to cancer.

Nearly all women with cervical cancer show signs of HPV infection on lab tests, but most women infected with HPV will not develop cervical cancer. Even though doctors can test women for HPV, there is no treatment directed at HPV itself. HPVs also have a suspected role in causing other cancers, including of the penis, anus, vagina, and vulva. They are likewise linked to certain cancers of the mouth and throat.

In other illustrative embodiments of the invention, Salicinium® is successfully employed as an anti-viral and/or immune-stimulating agent to treat a wide variety of additional viral infections, including, e.g., influenza, herpesviruses, Epstein-Barr virus (EBV), human T-cell leukemia virus-1 (HTLV-1) (causative agent of adult T-cell leukemia/lymphoma (ATL)), Raus sarcoma virus, Human papilloma viruses (HPVs), and Human Immunodeficiency Virus (HIV), among others).

Additional description relating to anti-cancer, anti-viral and immune-modulatory methods and compositions of the invention can be found, for example, in U.S. Continuation patent application Ser. No. 15/981,825, filed 16 May 2018; U.S. Continuation patent application Ser. No. 15/800,032, filed 31 Oct. 2017; U.S. Continuation patent application Ser. No. 15/469,532, filed 26 Mar. 2017; U.S. Continuation patent application Ser. No. 15/240,775, filed 18 Aug. 2016; U.S. Continuation patent application Ser. No. 14/981,895, filed 28 Dec. 2015; U.S. Continuation patent application Ser. No. 14/740,137, filed 15 Jun. 2015; U.S. Continuation patent application Ser. No. 14/583,087, filed 24 Dec. 2014; U.S. Continuation patent application Ser. No. 14/329,946, filed 13 Jul. 2014; U.S. Continuation patent application Ser. No. 13/907,951, filed 2 Jun. 2013; U.S. Continuation patent application Ser. No. 13/751,164, filed 28 Jan. 2013; U.S. Continuation patent application Ser. No. 13/525,317, filed 17 Jun. 2012; U.S. patent application Ser. No. 12/418,342, filed 3 Apr. 2009, which is entitled to priority from U.S. Provisional patent application, Ser. No. 61/042,210, filed 3 Apr. 2008, each of which herein by reference in its entirety for all purposes of description and discretionary priority.

The anti-cancer, anti-viral, and immune-stimulatory methods of the invention collectively involve administration of suitable, effective dosage amounts of Salicinium® to the treated subject. Typically, an effective amount will comprise an amount of the active compound (e.g., a glyco-benzaldehyde such as helicidum) which is therapeutically effective, in a single or multiple unit dosage form, over a specified period of therapeutic intervention, to measurably alleviate the targeted condition (such as, cancer or another proliferative disorder, viral infection, immune suppression). Within exemplary embodiments, cellular proliferative disorders including cancer and viral infections are effectively treated using a selected unit dosage of a benzaldehyde derivative compound of Formula I to V, or an intermediary or precursor compound of Formula VI or VII, which may be formulated with one or more pharmaceutically acceptable carriers, excipients, vehicles, emulsifiers, stabilizers, preservatives, buffers, and/or other additives that may enhance stability, delivery, absorption, half-life, efficacy, pharmacokinetics, and/or pharmacodynamics, reduce adverse side effects, or provide other advantages for pharmaceutical use.

Anti-cancer, anti-viral, and immune-stimulatory amounts of Salicinium® (e.g., a benzaldehyde derivative or compound of Formula I-V, or related, intermediary or precursor compound of Formula VI or VII) will be readily determined by those of ordinary skill in the art, depending on clinical and patient-specific factors. Suitable effective unit dosage amounts of the active compounds for administration to mammalian subjects, including humans, may range from 20 to 1000 mg, often with a minimum daily dose of 200-500 mg, and a maximum dose of 3,000-5,000 mg/day. In certain embodiments, the anti-cancer, anti-viral, and immune-stimulatory effective dose is between 500 to 4,000 mg/day, or about 2,000 or 3,000 mg/day. These and other effective unit dosage amounts may be administered in a single dose, or in the form of a multiple periodic dosing protocol, for example in a dosing regimen comprising from 1 to 5, or 2-3 doses administered per day, per week, or per month. In certain embodiments, the Salicinium active compound is dissolved in a solution for injection or intravenous (iv) delivery (e.g., a saline solution of 1-10%, as in the examples below using a 6% (3 grams/500 ml) Salicinium IV solution.

The amount, timing and mode of delivery of the anti-cancer, anti-viral, and immune-stimulatory compositions of the invention will be routinely adjusted on an individual basis, depending on such factors as patient weight, age, gender, and condition of the individual, the acuteness of the subject's disease and severity symptoms, whether the administration is prophylactic or therapeutic, prior treatment history (including e.g., any prior history and responsiveness to Salicinium® treatment) and on the basis of other factors known to effect drug delivery, absorption, pharmacokinetics, including half-life, and efficacy. An effective dose or multi-dose treatment regimen for the instant Salicinium formulations will ordinarily be selected to approximate a minimal dosing regimen necessary and sufficient to substantially prevent or alleviate the cancer, viral infection, immune disorder or other targeted condition, and/or to substantially prevent or alleviate one or more symptoms associated with that condition.

A dosage and administration protocol will often include repeated dosing therapy over a course of several days or even one or more weeks, up to several months, or even a year or more. An effective treatment regime may also involve prophylactic dosage administered on a daily or multi-dose per day basis lasting over a course of days, weeks, months or even a year or more.

Various assays and pre-clinical and clinical model systems can be readily employed to determine therapeutic effectiveness of the anti-cancer, anti-viral, and immune-stimulatory methods of the invention. For cancer, for example, these may detect/monitor a decrease in overt symptoms, such as pain (e.g., as measured using any of a variety of pain scales including, but not limited to, the Visual Analog Scale, McGill Pain Questionnaire, Descriptor Differential Scale, Faces Pain Scale, Verbal Rating Scale, Simple Descriptive Pain Scale, Numerical Pain Scale (NPS), or Dolorimeter Pain Index. More detailed detection/monitoring may document, for example, a decrease in circulating tumor cells (CTCs), reduction in tumor size, collapse or disappearance of tumors, softening of tumors, liquefaction of tumors, a decrease in cytological or histochemical cancer markers, among many other conventional diagnostic indicia of cancer disease stasis, progression and/or remission.

Effectiveness of the anti-cancer, anti-viral, and immune-stimulatory methods and compositions of the invention are demonstrated by a decrease in symptoms of cancer, viral disease and/or immunosuppression, which will typically involve a decrease of 5%, 10%, 25%, 30%, 50%, 75%, 90% or more in comparison to incidence/levels of the same symptoms in suitable control subjects (or known baseline or median data for like, treated or untreated subjects). For example, Salicinium-treated cancer patients will often exhibit a decrease in circulating tumor cells (CTCs) in successive blood assays during a course of Salicinium treatment, of from 25%-30%, 50%, 75% or higher, 90% and up to total absence of detectable CTCs. Monitoring of cancer, viral infection and immunosuppression symptoms can employ any of a vast array conventional detection and monitoring tools and indicia, as will be apparent to those skilled in the art. For example, CTC monitoring using blood samples of patients can utilize flow cytometry, immunobead capture, fluorescence microscopy, standard and density centrifugation, cell culturing, and immunocytochemistry. Similarly, tumor monitoring can employ x-ray, MRI, CT or PET scans, among other methods and tools. For economy these and other routine, well-known detection and monitoring technologies will not be reiterated here.

Effectiveness of immunotherapeutic treatment methods can likewise be determined via any of a diverse, well-known array of methods to monitor the status and activity of a subject's immune system. Such effectiveness may be demonstrated, for example by a decrease in secondary infections, an increase in immune cell count or activity (e.g., circulating numbers of macrophages, T cells, NK cells, B cells, or activity measures for these cells, such as immunoglobulin (Ig) levels, proliferation rates, motility or secretory activity (e.g., cytokine expression) assays, and the like.

Within additional aspects of the invention, combinatorial formulations and coordinate treatment methods are provided that employ an effective amount of a Salicinium® compound (e.g., a benzaldehyde derivative of Formula I-V, or precursor compound of Formula VI or VII) and one or more secondary or adjunctive agent(s) combinatorically formulated or coordinately administered with the Salicinium compound to yield an enhanced anti-cancer, anti-viral, and/or immune-stimulatory composition or method. Exemplary combinatorial formulations and coordinate treatment methods in this context employ a Salicinium compound in combination with the one or more secondary anti-cancer, anti-viral, and/or immune-stimulatory effective agents or drugs.

For treating cancer, exemplary combinatorial formulations and coordinate treatment methods employ a Salicinium compound in combination with one or more secondary or adjunctive anti-cancer effective agents, for example one or more chemotherapeutic agents selected from azacytidine, bevacizumab, bortezomib, capecitabine, cetuximab, clofarabine, dasatinib, decitabine, docetaxel, emend, erlotinib hydrochloride, exemestane, fulvestrant, gefitinib, gemcitabine hydrochloride, imatinib mesylate, imiquimod, lenalidomide, letrozole, nelarabine, oxaliplatin, paclitaxel, docetaxel, palifermin, panitumumab, pegaspargase, pemetrexed disodium, rituximab, sorafenib tosylate, sunitinib malate, tamoxifen citrate, targretin, temozolomide, thalidomide, and/or topotecan hydrochloride. Additional contemplated secondary anti-cancer effective agents in this context include, but are not limited to, interleukin-2, interferon α, filgrasten, G-CSF, epoetin alfa, erythropoietin, IL-1, oprelvekin, trastuzumab, vorinostat, antibiotics, coenzyme q, palladium lipoic complexes including, for example, poly-MVA®, antineoplastins, cartilage, hydrazine sulfate; milk thistle, electrolytes such as calcium carbonate, magnesium carbonate, sodium bicarbonate, and potassium bicarbonate; oxidizing agents, including, but not limited to, cesium chloride, potassium chloride, potassium orotate and potassium aspartate; immunoglobulins; colostrum; vitamin and mineral supplements including, but not limited to, zinc chloride, magnesium chloride, pyridoxine, vitamin B-12, B complexes, folic acid, sodium ascorbate, and L-lysine, probiotic compounds, Bacillus Calmette-Guerin vaccine, a non-corrosive base solution or alkaline water as described in U.S. patent application Ser. No. 12/167,123, filed Jul. 2, 2008 (incorporated herein by reference in its entirety), glutathione, grapeseed extract, columbianitin, and mistletoe extract. Additionally, adjunctive therapies may be used including, but not limited to, conventional chemotherapy, radiation therapy, surgery, alkaline water therapy (e.g., a pHenomenal® alkaline water regimen, see, e.g., U.S. patent application U.S. Continuation patent application Ser. No. 15/972,169, filed 6 May 2018; U.S. Continuation patent application Ser. No. 15/732,068, filed 12 Sep. 2017; U.S. Continuation patent application Ser. No. 15/421,744, filed 1 Feb. 2017; U.S. Continuation patent application Ser. No. 14/736,094, filed 10 Jun. 2015; U.S. Continuation patent application Ser. No. 14/526,433, filed 28 Oct. 2014; U.S. Continuation patent application Ser. No. 14/201,865, filed 9 Mar. 2014; PCT Patent Application No. PCT/US14/22204, filed 9 Mar. 2014; which is entitled to priority from U.S. Provisional patent application, Ser. No. 61/757,059, filed 25 Jan. 2013; and to U.S. Provisional patent application, Ser. No. 61/774,626, filed 8 Mar. 2013, each incorporated herein by reference in its entirety for all purposes), insulin potentiation therapy, the Gonzalez regimen, specialized anti-cancer diets, and acupuncture.

To practice coordinate administration methods of the invention, a Salicinium® composition (e.g., comprising helicidum or another glyco-toxin, such as another glyco-benzaldehyde compound) may be administered, simultaneously or sequentially, in a coordinate treatment protocol with one or more of the secondary or adjunctive therapeutic agents contemplated herein. Thus, in certain embodiments a Salicinium compound is administered coordinately with a non-Salicinium effective anti-cancer, anti-viral or immune-enhancing agent, using separate formulations or a combinatorial formulation. This coordinate administration may be done simultaneously or sequentially in either order, and there may be a time period while only one or both (or all) active therapeutic agents individually and/or collectively exert their therapeutic activities. A distinguishing aspect of all such coordinate treatment methods is that the Salicinium compound exerts at least some distinct therapeutic activity, yielding a distinct clinical response, in addition to any complementary clinical response provided by the secondary or adjunctive therapeutic agent. Often, the coordinate administration of a Salicinium compound with the secondary or adjunctive therapeutic agent will yield improved therapeutic or prophylactic results in the subject beyond a therapeutic effect elicited by the Salicinium compound or the secondary or adjunctive therapeutic agent administered alone. This qualification contemplates both direct effects, as well as indirect effects.

The Salicinium® pharmaceutical compositions of the present invention may be administered by any means that achieve their intended therapeutic or prophylactic purpose. Suitable routes of administration for the compositions of the invention include, but are not limited to, oral, buccal, nasal, aerosol, topical, transdermal, mucosal, injectable, intravenous and including all other conventional delivery routes, devices and methods.

The Salicinium® compounds of the present invention may be formulated with a pharmaceutically acceptable carrier appropriate for the particular mode of administration employed. Dosage forms of the compositions of the present invention include excipients recognized in the art of pharmaceutical compounding as being suitable for the preparation of dosage units as discussed herein. Such excipients include, without limitation, solvates, buffers, binders, fillers, lubricants, emulsifiers, suspending agents, sweeteners, flavorings, preservatives, wetting agents, disintegrants, effervescent agents and other conventional pharmaceutical excipients and additives.

Salicinium® compositions of the invention will often be formulated and administered in an oral dosage form, optionally in combination with a carrier and/or other additive(s). Suitable carriers for pharmaceutical formulation of oral dosage forms include, for example, microcrystalline cellulose, lactose, sucrose, fructose, glucose, dextrose, or other sugars, di-basic calcium phosphate, calcium sulfate, cellulose, methylcellulose, cellulose derivatives, kaolin, mannitol, lactitol, maltitol, xylitol, sorbitol, or other sugar alcohols, dry starch, dextrin, maltodextrin or other polysaccharides, inositol, or mixtures thereof. Exemplary unit oral dosage forms include ingestible and sublingual liquids, tablets, capsules, and films, among other options, which may be prepared by any conventional method known in the art, optionally including additional ingredients such as release modifying agents, glidants, compression aides, disintegrants, lubricants, binders, flavor enhancers, sweeteners and/or preservatives (e.g., stearic acid, magnesium stearate, talc, calcium stearate, hydrogenated vegetable oils, sodium benzoate, leucine carbowax, magnesium lauryl sulfate, colloidal silicon dioxide, glyceryl monostearate, colloidal silica, silicon dioxide, and glyceryl monostearate). Oral dosage forms may further include an enteric coating that dissolves after passing through the stomach, for example, a polymer agent, methacrylate copolymer, cellulose acetate phthalate (CAP), hydroxypropyl methylcellulose phthalate (HPMCP), polyvinyl acetate phthalate (PVAP), hydroxypropyl methylcellulose acetate succinate (HPMCAS), cellulose acetate trimellitate, hydroxypropyl methylcellulose succinate, cellulose acetate succinate, cellulose acetate hexahydro phthalate, cellulose propionate phthalate, cellulose acetate maleate, cellulose acetate butyrate, cellulose acetate propionate, copolymer of methyl methacrylic acid and methyl methacrylate, copolymer of methyl acrylate, methyl methacrylate and methacrylic acid, copolymer of methyl vinyl ether and maleic anhydride (Gantrez ES series), and natural resins such as zein, shellac and copal collophorium.

If desired, oral, mucosal, gastric, transdermal, topical and injectable compositions of the invention can be administered in a controlled release form by use of such technologies as slow-release carriers, controlled release agents in this context include, but are not limited to, hydroxypropyl methyl cellulose, having a viscosity in the range of about 100 cps to about 100,000 cps or other biocompatible matrices such as cholesterol.

In certain embodiments of the invention Salicinium® is administered to patients in an intravenous (iv) formulation and delivery mode. In illustrative aspects a therapeutic unit dosage of Salicinium (e.g., a representative glyco-benzaldehyde such as helicidum) is formulated in a physiologically buffered solution amenable for iv delivery to human subjects, typically an aqueous buffered solution, such as saline. This therapeutic iv formulation is administered over an effective iv delivery period, for example about 1-4 hours, by iv drip to the subject in a clinical setting. In exemplary embodiments, a simple saline formulation of 1-5 grams, typically about 3 grams in 500 ml of delivery solution was administered to subjects in multiple daily doses (e.g., 10-20, often about 15 separate daily iv administrations) over an aggressive iv treatment period of between about two weeks to about one month to six weeks, after which subjects are typically transitioned to an extended, oral Salicinium treatment protocol.

To illustrate an effective iv Salicinium® iv formulation, the following exemplary iv solution was manufactured to illustrate many optional ingredients that can be electively added to a base iv Salicinium formulation, to enhance therapeutic benefits beyond the fundamental anti-cancer, anti-viral and immune-enhancing benefits of a simple Salicinium iv solution. A Salicinium iv drip solution was prepared for anti-cancer therapy and immune-enhancement using 4 g of purified helicidum powder, initially mixed with 10 ml of 99.9% DMSO for solubilization. 1 ml of the resulting solution was injected into an iv-drip solution comprising 0.9% Sodium Chloride USP 0.9% (500 ml), 1,000 mg magnesium chloride, 1,000 mg Pyridoxine (B-6), 200 mg Vitamin B-12, 10 mg folic acid, 1,000 mg sodium ascorbate, 800 mg L-lysine, 25 mg zinc chloride, and 500 mg glutathione. This mixture was warmed above body temperature to ensure proper mixing, then cooled to body temperature before infusion into patients over a delivery period of 2 hours.

To illustrate manufacture of suitable oral dosage forms of Salicinium®, capsules containing a unit dosage form of the representative glyco-benzaldehyde were manufactured and prepared. Para-hydroxyl-benzaldehyde-O-B-D-allopyranoside was initially acetone extracted from crushed seeds of Helicia nilagirica, yielding 220 g of crude powder extract that was then placed in a 2 L beaker with 1,000 ml acetone, and this partially refined mixture was stirred and warmed with a condensing coil until it reached boiling temperature. The mixture was allowed to boil for 5 minutes and cooled. The warm mixture was filtered using Whatman #1 filter paper (Middlesex, U.K.) with a 1 L receiving flask and filter. The filter cake was washed two times with 250 ml proportions of acetone then vacuum dried. The filter cake was then cut into cubes and placed in a warm drying oven (60°−70° C.) until the acetone fully evaporated. Purity of the extract was determined by melting point analysis of the powder, which exhibited a melting point of 195/199° C. 200 g of the purified powder was then placed in a 600 ml beaker with 300 ml of 99% DMSO, then the solution was warmed to about 70° C. Once the powder was in solution, it was filtered using a vacuum filter through a 9 cm glass B\ddot{υ}chner funnel (Whatman GF/B filter) into a filter flask of 500 ml. The DMSO/powder solution was then poured into a 4 L beaker containing 3200 ml distilled water at 60-70° C. and stirred. The mixture was then cooled until crystallization began and finished in a refrigerator at 2°−5° C. for 18-24 hours. The cooled mixture was filtered through Whatman #1 paper and suctioned dry. The filter cake was then dried in a drying oven (70° C.) with a filtered air supply. The dried cake was then filtered through a U.S. series #10 stainless steel screen with an opening size of 78 thousandths of an inch.

An alternative, synthetic protocol for manufacturing a Salicinium® compound of the invention was exemplified for 4-O-b-D-glucopyranosylbenzldehyde. 5 grams of p-hydroxybenzaldehyde and 16.87 grams of tetra-O-acetyl-a-D-glucopyranosyl bromide were dissolved in 41 ml quinoline (acetonitrile may also be used in greater volume) and 5.4 grams of silver oxide was added slowly with stirring. After the exothermic reaction subsided, the stirring was maintained for 25 minutes while 27.5 ml glacial acetic acid was added. The resulting mixture was then poured into 1.2 liters of ice water. The resulting fine crystalline precipitate was then filtered with diatomaceous earth and the resulting filter cake washed with water. The washed filter cake was extracted with hot ethanol three times (250 ml each time). The ethanol extracts were concentrated in a partial vacuum which upon cooling and standing gave fine crystals, M. P. 143° C. The resulting compound was deacetylated with a slight molar excess of Sodium Methoxide in anhydrous Methyl alcohol to yield 4-O-b-D-glucopyranosyl benzaldehyde.

In more detailed embodiments, oral Salicinium® dosage forms may comprise, e.g., a benzaldehyde compound of Formula I-V or an intermediate or precursor compound of Formula VI or VII encapsulated for delivery in gelatin capsules, microcapsules, microparticles, or microspheres, prepared, for example, by coacervation techniques or by interfacial polymerization, for example, hydroxy methylcellulose or gelatin-microcapsules and poly(methylmethacylate) microcapsules, respectively, in colloidal drug delivery systems (for example, liposomes, albumin microspheres, microemulsions, nanoparticles and nano capsules), or within macroemulsions.

In other exemplary compositions and methods of the invention, Salicinium® is formulated topical administration, for example for direct topical treatment of skin cancer or Herpes virus lesions. Exemplary topical formulations are made using a glyco-benzaldehyde compound of Formula I-V, or an intermediate or precursor compound of Formula VI or VII, in combination with a topical delivery formulation additive, carrier, material or device. Topical compositions may, for example, incorporate the Salicinium compound in a dermatological or mucosally acceptable carrier, including in such forms as aerosol sprays, powders, dermal patches, sticks, granules, creams, pastes, gels, lotions, syrups, ointments, impregnated sponges, cotton applicators, or as a solution or suspension in an aqueous liquid, non-aqueous liquid, oil-in-water emulsion, or water-in-oil liquid emulsion. These topical compositions may comprise the Salicinium® compound dissolved or dispersed in water or another solvent, incorporated in a topical composition or device. Transdermal administration may be enhanced by incorporation of a dermal penetration or permeation enhancer, as are well known to those skilled in the art. Transdermal delivery may also be enhanced through techniques such as sonophoresis.

Yet additional Salicinium® compositions of the invention are designed for parenteral administration, for example for administration to patients intravenously, intramuscularly, subcutaneously or intraperitoneally. These formulations can include aqueous and nonaqueous sterile injectable solutions which, like many other contemplated compositions of the invention, may optionally contain anti-oxidants, buffers, bacteriostats and/or solutes which render the formulation isotonic with the blood of the subject, and aqueous and non-aqueous sterile suspensions which may include suspending agents and/or thickening agents. The formulations may be presented in unit-dose or multi-dose containers. Additional injectable compositions and formulations of the invention may include polymers for extended release following parenteral administration. The parenteral preparations may be solutions, dispersions or emulsions suitable for such administration. The Salicinium active compounds may be formulated with polymers for extended release. Parenteral Salicinium preparations will typically contain buffering agents and preservatives, often in a base of water, physiological saline, balanced salt solutions, aqueous dextrose, glycerol or the like. Extemporaneous injection solutions, emulsions and suspensions may be prepared from sterile powders, granules and tablets of the kind previously described. Preferred unit dosage formulations are those containing a daily dose or unit, daily sub-dose, as described herein above, or an appropriate fraction thereof, of the active ingredient(s). In some embodiments, localized delivery of Salicinium compounds (e.g., helicidum) may be achieved by injecting the parenteral formulation directly into an area surrounding a cellular malignancy, directly into a tumor, into the vasculature supplying a malignancy itself, or into a pleural or peritoneal cavity proximal to a targeted malignancy.

As noted above, in certain embodiments the methods and compositions of the invention may employ pharmaceutically acceptable salts, e.g., acid addition or base salts of a Salicinium® compound (e.g., selected from the above-described glyco-benzaldehyde and benzaldehyde derivative compounds). Examples of pharmaceutically acceptable addition salts include inorganic and organic acid addition salts. Suitable acid addition salts are formed from acids which form non-toxic salts, for example, hydrochloride, hydrobromide, hydroiodide, sulphate, hydrogen sulphate, nitrate, phosphate, and hydrogen phosphate salts. Additional pharmaceutically acceptable salts include, but are not limited to, metal salts such as sodium salts, potassium salts, cesium salts and the like; alkaline earth metals such as calcium salts, magnesium salts and the like; organic amine salts such as triethylamine salts, pyridine salts, picoline salts, ethanolamine salts, triethanolamine salts, cyclohexylamine salts, N,N′-dibenzyl ethylenediamine salts and the like; organic acid salts such as acetate, citrate, lactate, succinate, tartrate, maleate, fumarate, mandelate, acetate, dichloroacetate, trifluoroacetate, oxalate, and formate salts; sulfonates such as methane sulfonate, benzenesulfonate, and p-toluene sulfonate salts; and amino acid salts such as arginate, asparaginate, glutamate, tartrate, and gluconate salts. Suitable base salts are formed from bases that form non-toxic salts, for example aluminum, calcium, lithium, magnesium, potassium, sodium, zinc and diethanolamine salts.

In other detailed embodiments, the methods and compositions of the invention for employ Salicinium® prodrugs, e.g., prodrugs of a glyco-benzaldehyde Formula I-V, or of an intermediary compound, or precursor compound of Formula VI or VII. Prodrugs are considered to comprise a Salicinium compound reversibly linked (e.g., covalently bonded) to any carrier compound or moiety that functions to release the active Salicinium compound in vivo. Examples of prodrugs useful within the invention include esters or amides with hydroxyalkyl or amino alkyl as a substituent, among many other prodrug constructs known in the art.

The invention will also be understood to encompass methods and compositions comprising biologically active Salicinium® metabolites and in vivo conversion products (either generated in vivo after administration of the subject Salicinium compound, or directly administered in the form of the metabolite or conversion product itself). Such secondary active products may result for example from oxidation, reduction, hydrolysis, amidation, esterification and the like, of the administered compound, primarily due to enzymatic processes.

Accordingly, the invention includes methods and compositions of the invention employing compounds produced by a process comprising of contacting a Salicinium compound (e.g., a benzaldehyde compound of Formula I-IV or intermediate or precursor compound of Formula VI or VII) with a mammalian cell, tissue, body fluid or physiological compartment for a period of time sufficient to yield a metabolic product thereof. Such products typically are identified by preparing a radiolabeled compound of the invention, administering it parenterally in a detectable dose to an animal such as rat, mouse, guinea pig, monkey, or man, allowing sufficient time for metabolism to occur and isolating labeled conversion products from the urine, blood or other biological samples.

The invention disclosed herein will also be understood to encompass diagnostic compositions for diagnosing the risk level, presence, severity, or treatment indicia of, or otherwise managing a malignant disease, viral infection or immune disorder in a mammalian subject. In general terms these methods and compositions may employ a labeled compound (e.g., isotopically labeled, fluorescent labeled or otherwise labeled) to permit detection of the labeled after delivery to a mammalian subject (e.g., to a cell, tissue, organ, or individual) at risk or presenting with one or more symptom(s) of malignancy, viral infection or immune disorder, and thereafter detecting the presence, location, metabolism, and/or binding state (e.g., detecting binding to an unlabeled binding partner involved in glyco-benzaldehyde receptor physiology or metabolism) of the labeled compound using any of a broad array of known assays and labeling/detection methods.

### Example I

### Salicinium Induces Apoptosis in Circulating Cancer Cells Harvested from Human Cancer Patients

Circulating tumor cells (CTCs) were isolated and characterized from patients with diverse diagnoses of cancer (including a wide variety of primary cancer types, with and without metastasis) using conventional flow cytometry modified to a multiparameter flow cytometric panel employing magnetic bead separation methods to characterize CTCs and isolate them from patient blood samples. In one exemplary modified procedure for isolating and characterizing CTCs from breast cancer patients, the flow cytometry panel included CD45-PE/Cy7, CD31− RPE, pan cytokeratin-PE/Cy5, c-met-PE and MUC-1(CD227)-FITC. CTCs were identified as CD45−/CD31−/PanCK+/MUC1+ and metastatic cells as CD45−/c-met+. CTC isolation and cultivation utilized PBMCs from patients isolated using ficoll centrifugation methods and incubated with EpCAM magnetic beads to isolate the CTCs. Other procedures were adapted using comparable flow cytometric panels adapted for different cancer types according to known methods (see for example, Pantel et al., Detection and characterization of residual disease in breast cancer. J Hematother 3:315-22, (1994); Radbruch et al., Detection and isolation of rare cells. Curr Opin Immunol 7:270-3 (1995); and Ma et al., Predictive value of circulating tumor cells for evaluating Short- and Long-Term efficacy of chemotherapy for Breast Cancer. Med Sci Monit 23:4808-4816 (2017), each incorporated herein by reference).

Isolated CTCs were cultured in serum free RPMI medium. Test samples were exposed to Salicinium® (represented here by an aqueous solution of helicidum added to test cultures to achieve a test concentration of helicidum of 0.5 mg/ml in the samples) and incubated 24 hours before microscopic observations were made in replicate series to observe and quantify apoptosis in the CTC samples. Apoptosis quantification was based on observed cytoplasmic, nuclear and membrane changes diagnostic for apoptosis. In early stage of apoptosis, cells expand and become round, detach from adjacent cells or substrate and shrink. In the cytoplasm, the endoplasmic reticulum expands and turns vacuolar. In the nucleus, chromatin condenses in a crescent form around the nuclear membranes and becomes more basophilic. Eventually the nucleus fragments and cells convert to apoptotic bodies in containing fragmented nuclear and cellular materials. In vivo these apoptotic bodies are recognized and digested by phagocytes without raising inflammatory responses.

Data were analyzed using SPSS software, and T test methodology was used to compare data sets. A significance level of p<0.05 was considered statistically significant.

From these studies, CTCs were isolated with high fidelity and shown to be powerful tools to monitor cancer disease progression in individual patients, and more particularly for determining efficacy of anti-cancer drugs and methods against patient-specific samples.

A total of 967 patient CTC samples were tested within this study, and of these samples 82% showed statistically significant sensitivity to Salicinium for inducing apoptosis in the cultured CTC cells (18% of samples showed no detectable apoptotic activity, comparable to control samples).

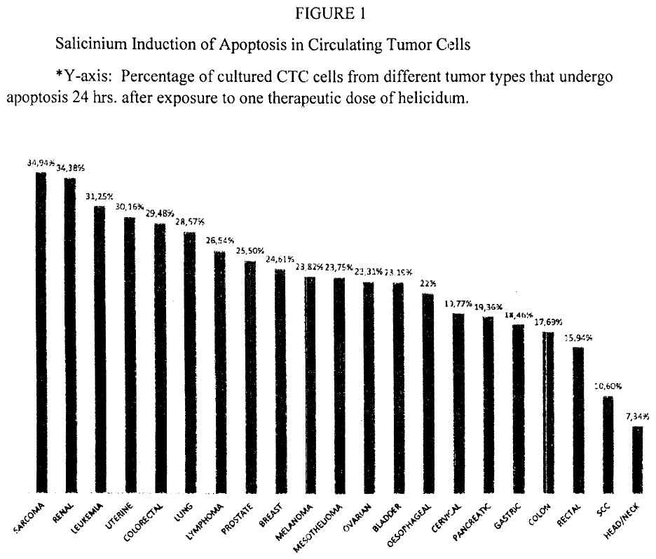

As illustrated in FIG. 1, Salicinium potently induced CTC apoptosis in virtually all cancer types. For more sensitive cancer types, including lung, colorectal, sarcoma and renal cancer, a single dose (comparable to a clinical therapeutic dose as described below) of Salicinium induced apoptosis in approximately 30-35% of all CTC cells present in the sample. For the majority of other cancer types tested, Salicinium effectively induced apoptosis in about 20-25% of CTC cells in samples after a single exposure and 24-hour test period. Even less sensitive cancers, for example squamous cell carcinoma (SCC) and head/neck cancer, showed very potent induction of apoptosis (70-10%) predictive of a profound therapeutic benefit over multiple treatments.

Additional studies evaluated caspase levels in CTC samples treated or untreated with Salicinium. Caspases are major executants of apoptosis. They are cysteine proteases that are generally inactive in healthy cells. During apoptosis these pro-enzymes are converted into active enzymes which mediate apoptosis in part by degrading intracellular proteins, for example cytoskeletal proteins, causing profound morphological changes of cells. Caspase-3 is activated by upstream caspases (caspase-8, caspase-9 or caspase-10), and in turn Caspase-3 activates endonuclease CAD (caspase activated DNase). In proliferating cells, CAD normally combines with ICAD (an inhibitor of CAD) to form an inactive complex. In apoptosis, ICAD is cut by caspase-3 and release CAD, followed by rapid fragmentation of DNA.

Caspase-9 levels were compared in control CTC samples and Salicinium-treated CTC samples according to conventional assay methods. Commensurate with the observed induction of apoptosis by Salicinium, we observed potent induction of elevated caspase-9 levels in Salicinium-treated versus control samples of diverse CTCs from different cancer types.

### Example II

### Salicinium Therapy Prolongs Five Year Survival in Stage IV Cancer Patients

A ten-year, multi-center cancer study was conducted wherein we recruited a total of 675 Stage IV cancer patients. These patients were treated and closely monitored through four participating medical and naturopathic clinics, with each clinic following standard therapeutic, monitoring and reporting protocols for each patient over a minimum study period of five years. The patients in these studies were all diagnosed by standard diagnostic methods (e.g., using tumor visualization, cytology, biopsy, blood assays for circulating tumor markers, etc.) at the start of the study as having Stage IV cancer, according to conventional diagnostic standards. Cancer types were grouped by primary cancer cytology/histochemistry, patient history and other standard criteria.

Of the 675 enrolled patients, 128 patients entered the study with diagnosed Stage IV breast cancer, 91 with Stage IV colon/rectal cancer, 34 with Stage IV head/neck cancer, 86 with Stage IV lung cancer, 32 with Stage IV non-Hodgkin's lymphoma (NHL), 28 with Stage IV melanoma, 36 with Stage IV ovarian cancer, 37 with Stage IV pancreatic cancer, 76 with Stage IV prostate cancer, 18 with Stage IV renal cancer, 23 with Stage IV sarcoma, and 34 with Stage IV uterine cancer.

Many of the enrolled subjects in this clinical study presented with Stage IV cancer that had persisted for many months, and in many cases years, following a prior Stage IV cancer diagnosis, and after having undergone one or more failed rounds of conventional oncotherapy (typically a combination of surgery, chemotherapy, radiation and/or hormonal therapy as indicated). These subjects are classified as “non-responsive” to conventional oncotherapy. A majority of the remaining study subjects enrolled in our study after an original Stage III or Stage IV cancer diagnosis, followed by conventional oncotherapy prior to the study, and presented at the time of study enrollment with disease progression to Stage IV cancer or with relapse of a prior, treated cancer. These subjects are classified as “treatment resistant” or “refractory” Stage IV cancer patients. For all Stage IV cancer study sub-groups enrolled here, only 5-10% of the group participants had not received prior, conventional oncotherapy. Accordingly, the study groups presented here are qualified for each cancer type at the time of enrollment, as representing a class of treatment-resistant, refractory or non-responsive Stage IV cancer patients (i.e., having had previous, conventional oncotherapy which failed to prevent progression or relapse of their disease).

Many of our subjects with confirmed Stage IV cancer diagnoses at the start of our study continued some course of conventional oncotherapy during the Salicinium treatment/study period. All patients remained throughout the study period in contact with medical providers who maintained regular monitoring of the subject's disease condition (with regular exams, including blood work testing for cancer markers, cytology, CT scans, PET scans, biopsy where indicated, and other diagnostic monitoring). Many of the study patients, while classified as having “treatment-resistant” Stage IV disease, nonetheless continued with some form of conventional oncotherapy (e.g., low dose chemotherapy) as recommended by their oncologist. Of the enrolled subjects in this study, 52% maintained some form of chemotherapy, or initiated some form of chemotherapy during the course of the instant study. About half of these subjects received conventional dose chemotherapy at some point, while the remainder received “low dose chemotherapy” (e.g., using a 10-15% dosing of a standard chemotherapeutic drug such as a taxane). Likewise, among study subjects who received some follow-on, conventional treatment during the instant study, 26% of enrolled subjects received one or more surgical or radiation treatments at some point during the course of the study. Additionally, patients with breast cancer, ovarian cancer, uterine cancer and prostate cancer remained on any oncologist-prescribed hormonal therapy.

Patients in each study group were treated with a standard Salicinium® therapy regimen as described herein. Each patient was provided an initial aggressive treatment regimen of intravenous (iv) Salicinium® therapy, comprising 15 iv treatment sessions administered over a period of 15 days to one month (depending on patient availability, travel and other scheduling requirements). Patients were administered 500 ml iv Salicinium comprising a 0.06% solution (3.0 g of 4-(beta-D-allopyranosyloxy)-benzaldehyde (helicidum) in 500 ml saline) over a two-hour administration period each day, typically scheduled over 3 blocks of 5-day treatment periods with two-day intermissions (with oral dosing of Salicinium® as described below on non-iv days) for most subjects. In certain cases, all 15 iv treatments were administered over a consecutive 15-day period, while in others the 15 iv treatments were scheduled over as much as a one-month period to accommodate patient scheduling factors.

After the initial aggressive iv treatment period, all patients were switched to oral Salicinium® treatment, specifically a regimen comprising a 3 g/day dosing protocol carried out for one year or until a subject was evaluated to be in substantial disease remission. Subjects self-administered three 500 mg gelatin capsules containing Salicinium (in this study, 500 mg powder form 4-(beta-D-allopyranosyloxy)-benzaldehyde) twice daily between meals. Subjects who did not progress rapidly to partial or complete remission remained on the oral Salicinium dosing regimen for one month following their positive diagnosis of remission, while other subjects remained on the oral treatment for the full treatment period of one year.

All subjects were regularly examined for diagnostic cancer indicators, assessed according to standard methods. Typically, this involved monthly, quarterly or bi-annual examinations by their regular oncologist, yielding study data in accordance with standard oncological monitoring procedures (e.g., CT scans, PET scans, blood work, cytology, biopsy, etc., as indicated for the subject patient consistent with their original diagnosis, last evaluated disease state, and current symptomology).

All patients who did not reach a state of remission within the first year completed the full, one-year oral Salicinium dosing regimen. Certain patients who presented with high-risk diagnostic indicators (e.g., new tumors or actively growing tumors by CT and/or PET scans, high levels of cancer blood markers, etc.) continued oral Salicinium® as long as these risk indicators remained high, though for most patients the oral treatment was determined to be complete/successful by one year-18 months.

Based on these cumulative studies and data generated for the 675 study subjects in our clinical investigation, the anti-cancer therapeutic efficacy of our novel Salicinium® treatment protocol has now been unambiguously demonstrated. As the data summarized in Table 2 reveal, the efficacy of Salicinium is no less than extraordinary for its ability to transition patients from intractable, Stage IV cancer to stable disease, partial remission, or complete remission, with few or no reported adverse side effects (apart from side effects attributable to conventional oncotherapy in patients who combined Salicinium® and conventional therapy).

TABLE 1
Cumulative Salicinium ® Clinical Study Results, by
Patient Status Through End of Five-Year Study Period

| Cancer | Number of | Stable | Partial | Complete | Not |
|---|---|---|---|---|---|
| Type | Patients Studied | Disease | Remission | Remission | Surviving |
| Breast | 128 | 38 | 30 | 21 | 39 |
| Colon/Rectal | 91 | 36 | 15 | 5 | 35 |
| Lung | 86 | 38 | 10 | 5 | 33 |
| Prostate | 76 | 22 | 23 | 15 | 16 |
| Melanoma | 29 | 7 | 8 | 0 | 14 |
| Non-Hodgins | 32 | 6 | 5 | 6 | 15 |
| Lymphoma | | | | | |
| Ovarian | 36 | 11 | 5 | 4 | 16 |
| Uterine | 34 | 10 | 4 | 2 | 18 |
| Head/Neck | 34 | 14 | 0 | 4 | 16 |
| Renal | 18 | 7 | 0 | 3 | 8 |
| Sarcoma | 23 | 7 | 2 | 2 | 12 |
| Pancreatic | 37 | 17 | 0 | 0 | 20 |

The clinical study results shown here prove that Salicinium® (e.g., glyco-benzaldehyde compounds represented by helicidum) potently stabilizes and frequently eradicates cancer in the most intractable Stage IV, treatment-resistant and non-responsive subjects.

As compared to published, median five-year survival rates (for all classes of Stage IV patients, combining all known treatment modalities), the results here are beyond surprising. For example, we observed an astounding 69% five-year survival rate among Salicinium-treated Stage IV breast cancer subjects. This contrasts starkly with the median five-year survival rate of only 16% published by the National Institutes of Health (NIH) for this unfortunate class of cancer patients. These observations reveal a greater than four-fold increase in five-year survival expectancy (from 16% to 69%) for Salicinium-treated these patients. 16% of the 128 enrolled Stage IV breast cancer patients were essentially cured to a status of “complete remission” (with no cancer discernable by any conventional diagnostic measure), while an additional 53% of all enrolled subjects were classified as having “stable disease” or being in “partial remission” at the end of the five-year study (Table 1).

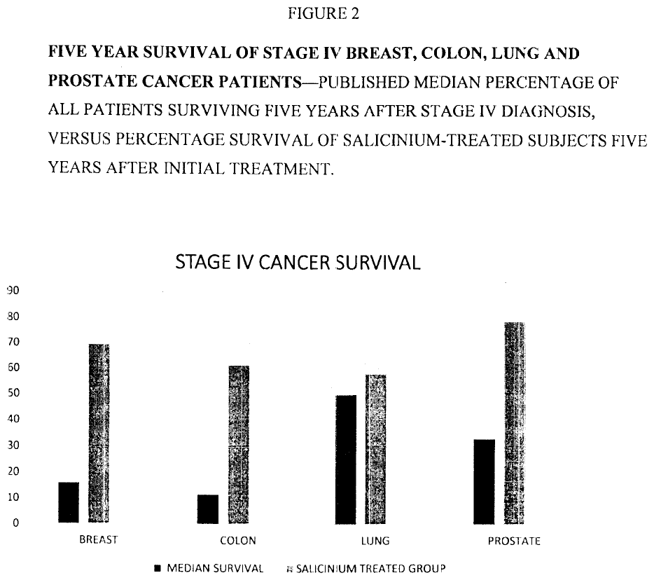

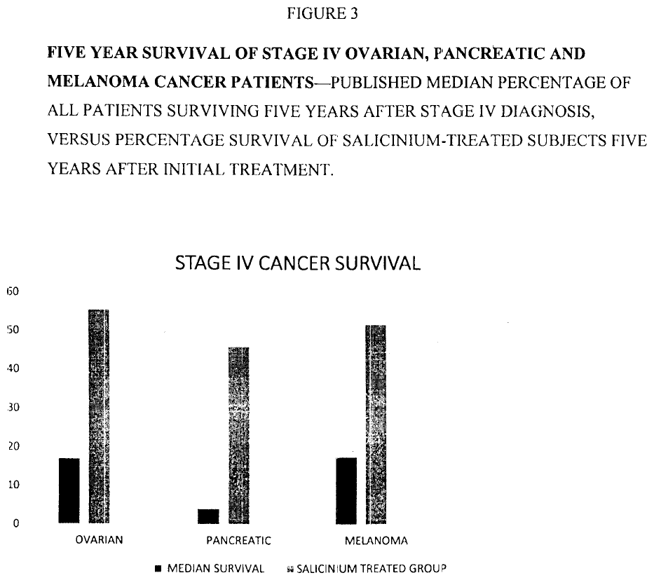

Comparable survival improvement was demonstrated for all classes of Salicinium-treated subjects in our study. Exemplary data relating to the most common, costly and fatal cancer types are provided in FIGS. 2 and 3 below, which illustrate a stunning contrast in survival expectancy between median Stage IV cancer survival data compiled by the NIH, and data determined here for Salicinium-treated patients. Thus, among 91 colon/rectal cancer patients who completed Applicant's clinical study, 5% achieved total remission, while an additional 56% survived with stable disease or partial remission. This demonstrates a total survival percentage of 61%, more than five-times greater than the 8-15% median survival (average 11.5%) published by the NIH (Table 1, FIG. 2).

For lung cancer, Salicinium®-treatment following Applicants' teachings yielded 62% five-year survival (FIG. 2). This marks more than a 10% increase in survival expectancy compared to NIH published data, which relatively modest improvement reflects an unusually high survival rate for Stage IV lung cancer patients in general (NIH published median survival is 50% for this group), and a relatively low total remission rate (6% with 56% reported as stable or in partial remission) for lung patients among the Salicinium results reported here for other types of cancers (Table 1).

Prostate cancer results from Salicinium treatment in our study were particularly positive, with 78% survival—more than double the NIH published median survival of 33% (FIG. 2). Additionally, in the prostate cancer study group there was a particularly potent clinical benefit of Salicinium with respect to mediating total disease remission, which was observed in 20% of these study subjects (Table 1). An additional 58% of the prostate cancer study population survived in stable or partial remission status.

For the Stage IV melanoma study group, Salicinium treatment mediated an unexpectedly high, 52% five-year survival outcome (compared to NIH published median survival for all types of Stage IV skin cancer, of 15-20%) (FIG. 3). Taking the average of the NIH compiled figures of 17.5%, this marks about a three-fold increase in patient survival over median survival attributable to the Salicinium treatment. All of the surviving members of the melanoma study group ended the study classified as stable or in partial remission (Table 1).

The Stage IV pancreatic cancer study group showed the most marked increase in survival results compared to the grim, 4% median five-year survival expectancy as published by the NIH. After treatment with our novel Salicinium regimen, 76 study subjects showed a 46% survival rate, or more than 11 times the median survival expectancy (FIG. 3). Here again it is important to note that the median survival statistics comprehend the results of all known, conventional cancer treatments (albeit, these statistics also include a fractional component of individuals who never receive treatment). As in the melanoma group, all surviving members of the pancreatic cancer study group were classified as stable or in partial remission at the end of the five-year study (Table 1).

With regard to the Stage IV ovarian cancer group these subjects exhibited an overall survival rate of 55%, more than three times the median survival of 17% for Stage IV ovarian cancer published by the NIH (FIG. 3). More than 10% of these subjects also completed the study classified in complete remission.

Prolongation of survival was similarly observed for all other Stage IV cancer study groups. Five-year survival observed for non-Hodgkin's lymphoma subjects was 53%, with 34% of study subjects classed at the end as stable or in partial remission, and a compelling 19% of subjects in total remission. For uterine cancer subjects the observed survival was 47% (41% stable or partial remission, 5% total remission). For cancer patients with particularly challenging.

Stage IV head/neck cancer, survival was 53%, with 41% of all study subjects classed as stable or in partial remission, and 12% presenting at the end of the study in complete remission (Table 1). For renal cancer subjects, 56% survived the five-year study period, 17% in total remission and 39% diagnosed as stable or in partial remission. For sarcoma subjects, the results were also unexpectedly positive, with 48% survival (9% total remission and 39% observed to be stable or in partial remission).

In addition to the 624 patients treated and evaluated over the five-year study whose results are summarized in Table 1, the instant also study included 51 additional patients presenting with ALL, AML, astrocytoma, bladder cancer, CLL, esophageal cancer, gall bladder cancer, stomach cancer, glioblastoma, Hodgkin's disease, liver cancer, mesothelioma, myeloma, testicular cancer and thyroid cancer. In all of these smaller treatment/study groups the methods and compositions of the invention achieved significant therapeutic benefits over a five-year treatment and monitoring period, in terms of increased rates of stable disease, remission and survival, consistent with the larger study groups evaluated.

### Example III

### Salicinium Potently Disrupts Nagalase Expression and Reduces Nagalase Blood Levels in Cancer Patients and Patients with High Pathogenic Viral Loads, Causing an Associated Increase in Immune Function and Potent Immune Destruction of Cancer and Viral-Infected Cells

To further illustrate the role of Salicinium® in fighting cancer and viral infection through disruption of alpha-N-acetylgalactosaminidase (nagalase), we assayed pre- and post-treatment nagalase levels in blood samples from 158 patients. These patients were selected as having been diagnosed with a heavy load, chronic viral infection and/or Stage IV cancer. Some of these subjects presented with only viral infection, and some with only Stage IV cancer, whereas a sizeable group of 64 subjects had co-morbid Stage IV cancer attended by a heavy load chronic viral infection. The principal viral subjects in these studies were Epstein Barr virus (EBV), hepatitis C virus, cytomegalovirus (CMV) and herpes virus.

Nagalase levels and viral load were quantified using conventional assays for each patient, before and after successive iv Salicinium® treatments, and after extended oral Salicinium follow-on therapy (using the iv helicidum iv and oral treatment protocol described in Example II, above).

Data obtained within this study show that nagalase levels are typically very elevated in Stage IV cancer patients. In particular, whereas we have determined a conditional “normal” nagalase level as corresponding to about 0.65 Units (nmol/min/mg), our stage IV cancer subjects exhibited elevated nagalase levels routinely above 0.95 units, often between about 1.2-2.5 Units. Extreme outlier patients, with the most severe and pervasive cancers, exhibited extraordinarily high nagalase levels, up to 4.0 Units and even higher. On average, our Stage IV cancer subjects evaluated in this study for Salicinium impacts on nagalase blood levels tested with a median nagalase blood level of 1.43 Units at the time of enrollment, prior to the initial Salicinium treatment. This study group of 158 patients was followed up for nagalase assays every month for the first three months after treatment, again at six months, and again at one-year post treatment. The cumulative data from these studies showed that iv Salicinium treatment effectuated pronounced reductions in nagalase levels in a large majority (83%) of patients over the first three monthly post-treatment checkpoints, so that by one month after treatment median nagalase levels in these subjects was decreased from a starting value of 1.43 Units to about 1.15 Units. By the third month median nagalase levels in these subjects was decreased to about 1.12 Units. By six months, 72% of the study patients exhibited nagalase levels below 1.0 units, and by one year 86% of study patients exhibited nagalase levels in a conventionally accepted “normal” range of below 0.95 Units. The median nagalase level determined at 1-year post-treatment was 0.78 Units.

For the cancer group, all of these data showed strong relationship to the severity of individual patient's initial cancer status. Patient's initiating the study with large involved cancerous tissue volumes expressed the highest nagalase levels, and showed a more gradual percentage reduction in nagalase levels over time. In contrast, patients with smaller tumors and less pervasive forms of cancer (e.g., prostate and breast cancer, versus high load skin cancer) showed lower initial nagalase levels and a more rapid recovery to normal levels.

Conventional viral load assays showed parallel anti-viral efficacy of Salicinium® closely tracking the nagalase attenuation data presented above. For EBV, hepatitis C, CMV and herpes II virus in particular, median viral loads among study subjects for all of these viruses dropped by about 20-25% in the first month, and by an additional 20% within three months after treatment. The most compelling data related to total viral eradication numbers (i.e., no detectable virus in the subjects). For EBV and herpes II subjects, initial high load viral infections became undetectable in 50% of treated subjects within six months following initial Salicinium iv treatment (supported by oral Salicinium maintenance treatment as described in Example II). After one year, 87% of all viral subjects (presenting with EBV, hepatitis C, herpes virus, and CMV) were entirely clear of detectable virus.

### Immune Activation Effects of Salicinium on Natural Killer (NK) Cells from Cancer Patients

Immune cells were obtained from blood samples of 73 cancer patients. The capability of these immune cells to kill tumor cells in vitro was determined using a conventional cellular NK activity assay (Neri et al, Clin. Diagn. Lab. Immunol. 2001 November; 8(6): 1131-1135). Basal killing activity of NK cells was compared to killing activity after exposure of test samples to a therapeutic concentration of Salicinium®Isolated immune cells were cultured in serum free RPMI medium.

Test samples were exposed to Salicinium® (helicidum added to test cultures to 0.5 mg/ml in the samples) and incubated 24 hours before microscopic observations were made in replicate series to observe and quantify NK cell destruction of tumor cells in the samples. Results were determined as percent NK cell-mediated lysis in control samples (tumor cells killed in the absence of Salicinium) and treatment samples (tumor cells killed with Salicinium present).

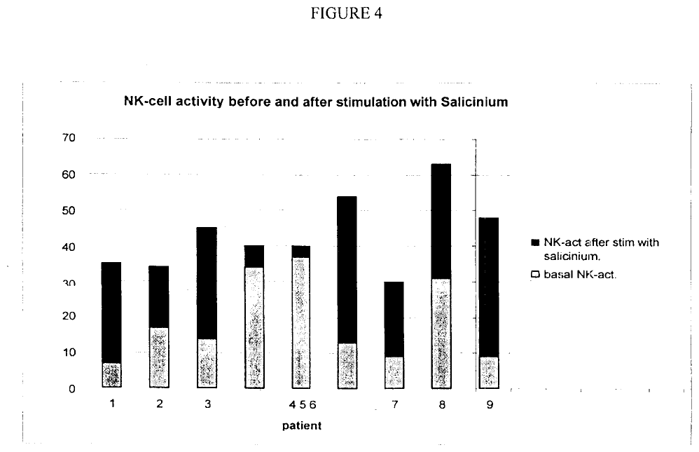

A total of 73 patient samples were evaluated in this manner, before any Salicinium® treatment regimen was initiated for the patient. Control samples for these assays employed immune cells from patients diagnosed as having no detectable cancer. One exemplary test run (FIG. 4) compared nine patient samples side by side (samples 4 and 5 are controls, from patients with no cancer). As can be readily seen, these data show that Salicinium mediates a major increase in NK cell cancer-killing activity, yielding NK activation (above basal activity levels) increases of from about 50% to 2- or 3-fold, up to about 4-fold or higher.

In the cumulative samples tested, the average Salicinium®-mediated increase in NK cell cancer-killing activity was approximately 2- or 3-fold (average 2.6 times greater) compared to basal NK cell cancer-killing activity. Controls routinely showed little or no increase in NK cell activity in the presence of Salicinium. The interpretation of these data follows our findings demonstrated here that cancer effects an upregulation of nagalase, which suppresses GcMAF and thereby suppresses macrophage activation and macrophage-mediated stimulation of downstream immune functions, including NK cell activation. This results in suppression of circulating immune cells in patients with cancer, and when these cells are harvested and cultured, as in these studies, in the presence of Salicinium®, the immunosuppression mediated by nagalase is reversed over a culture period wherein Salicinium disrupts nagalase synthesis by the cancer cells and the titer of nagalase in the test cultures drops dramatically, relieving the inhibition of GcMAF and NK function in those cultures. In control cultures, no such inhibition is present initially, due to normal nagalase levels, and so the dramatic increase in NK cell activity is not observed and baseline NK cell activity is much higher.

The invention is described herein for illustrative purposes and is not limited by the description herein. Rather, the inventors claim all embodiments of cancer treatment and prevention methods and compositions, immune-modulatory methods and compositions, and methods and compositions for diagnosing, managing, treating and preventing other proliferative orders, such as various hyperplasias, endometriosis, psoriasis, blood proliferative disorders, and other aberrant cellular and tissue proliferative conditions.

Although the foregoing invention has been described in detail by way of example for purposes of clarity of understanding, it will be apparent to the artisan that certain changes and modifications may be practiced within the scope of the appended claims which are presented by way of illustration not limitation. In this context, various publications and other references have been cited with the foregoing disclosure for economy of description. Each of these references is incorporated herein by reference in its entirety for all purposes.

We claim:

1. A method for inducing apoptosis in a circulating tumor cell (CTC) population, cancerous tissue or cancerous tumor in a mammalian subject, sufficient to prevent progression of a cancer disease condition in the subject, comprising administering a cancer apoptosis-inducing effective amount of a glyco-benzaldehyde compound in an oncotherapeutic treatment protocol, sufficient to induce disease-stabilizing, disease-reducing or disease-eliminating apoptosis in the CTC population, cancer tissue or tumor in the subject.

2. The method of claim 1, which is effective to extend a five-year survival rate among Stage III or Stage IV cancer patients (compared to median survival within a comparable group of conventionally-treated or untreated cancer patients) by at least 10%.

3. The method of claim 1, which is effective to extend a five-year survival rate among Stage III or Stage IV cancer patients (compared to median survival within a comparable group of conventionally-treated or untreated cancer patients) by at least 15%.

4. The method of claim 1, which is effective to extend a five-year survival rate among Stage III or Stage IV cancer patients (compared to median survival within a comparable group of conventionally-treated or untreated cancer patients) by at least 25%.

5. The method of claim 1, which is effective to extend a five-year survival rate among Stage III or Stage IV cancer patients (compared to median survival within a comparable group of conventionally-treated or untreated cancer patients) by 30-50% or greater.

6. The method of claim 1, which is effective to extend a five-year survival rate among Stage III or Stage IV cancer patients diagnosed with a treatment-resistant cancer selected from treatment-resistant breast cancer, lung cancer, prostate cancer, skin cancer including melanoma, liver cancer, thyroid cancer, esophageal cancer, sarcoma, brain cancer of all types, colon and rectal cancers, bladder cancer, gall bladder cancer, stomach cancer, renal cancer, ovarian cancer, uterine cancer, cervical cancer, non-Hodgkin's lymphoma, acute myelogenous leukemia (AML), acute lymphocytic leukemia, chronic lymphocytic leukemia (CLL), myeloma, mesothelioma, pancreatic cancer, Hodgkin's disease, testicular cancer, Waldenstrom's disease, head/neck cancer, cancer of the tongue, and/or malignancies induced by SV₄₀ virus (compared to median survival within a comparable group of treatment-resistant cancer patients, receiving no further therapy or continuing to receive conventional oncotherapy) by at least 25%.

7. The method of claim 1, wherein the glyco-benzaldehyde compound is a compound of Formula I, below, or an analog of Formula I having the illustrated glycome moiety substituted by: 1) any α or β form hexose selected from mannose, galactose, or fructose; or 2) a biose formed from two or more hexoses wherein the hexoses may be the same or different.

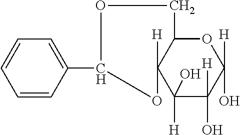

8. The method of claim 1, wherein the glyco-benzaldehyde compound is selected from 4, 6-0-benzylidine-D-glucopyranosyloxy, 2-β-D-glucopyranosyloxy benzaldehyde, 3-β-D-glucopyranosyloxy benzaldehyde, and 4-β-D-glucopyranosyloxy benzaldehyde.

9. The method of claim 1, wherein the glyco-benzaldehyde compound is administered to said subject in an anti-cancer effective dosage of from about 500 to about 4000 mg per day.

10. The method of claim 1, wherein the glyco-benzaldehyde compound is administered to said subject in an anti-cancer effective, intravenous dosage form comprising an aqueous solution of from about 2,000 to about 5,000 mg per day.

11. The method of claim 1, wherein the glyco-benzaldehyde compound is administered to said subject in an anti-cancer effective, intravenous (iv) dosage form comprising an aqueous solution of from about 2,000 to about 5,000 mg per day, wherein the subject is administered at least ten of these iv treatment doses within a first month of treatment.

12. The method of claim 1, wherein the glyco-benzaldehyde compound is administered to said subject in an anti-cancer effective, intravenous (iv) dosage form comprising an aqueous solution of from about 2,000 to about 5,000 mg per day, wherein the subject is administered 10-15 of these iv treatment doses within an initial three-week aggressive treatment period.

13. The method of claim 1, wherein the glyco-benzaldehyde compound is administered to said subject in an anti-cancer effective, intravenous (iv) dosage form comprising an aqueous solution of from about 2,000 to about 5,000 mg per day, wherein the subject is administered 10-15 of these iv treatment doses within an initial one month aggressive treatment period, followed by maintenance treatment with an oral dosage form comprising about 500 to about 4000 mg of the glyco-benzaldehyde compound formulated for oral delivery, per day.

14. The method of claim 1, wherein the glyco-benzaldehyde compound is helicidum administered to said subject in an anti-cancer effective, intravenous (iv) dosage form comprising an aqueous solution of from about 2,000 to about 5,000 mg per day, wherein the subject is administered approximately 15 iv doses within an initial three week aggressive treatment period, followed by maintenance treatment with an oral dosage form comprising about 1,000 to about 3,000 mg of the glyco-benzaldehyde compound formulated for oral delivery, per day.

15. The method of claim 1, which is effective to decrease tumor count in said subject by 10%-90% or greater over an effective course of treatment.

16. The method of claim 1, which is effective to decrease average tumor size in said subject by 10%-90% or greater over an effective course of treatment.

17. The method of claim 1, which is effective to decrease a circulating tumor cell (CTC) count in serial blood samples of said subject by 10%-90% or greater over an effective course of treatment.

18. The method of claim 1, which is effective to achieve total remission (with no tumors or cancer blood markers detectable by any conventional cancer diagnostic method) in at least 15% of stage IV cancer patients within two-six months following initial treatment.

19. The method of claim 1, which is effective to treat one or more cancer forms selected from breast cancer, lung cancer, prostate cancer, skin cancer including melanoma, liver cancer, thyroid cancer, esophageal cancer, sarcoma, brain cancer of all types, colon and rectal cancers, bladder cancer, gall bladder cancer, stomach cancer, renal cancer, ovarian cancer, uterine cancer, cervical cancer, non-Hodgkin's lymphoma, acute myelogenous leukemia (AML), acute lymphocytic leukemia, chronic lymphocytic leukemia (CLL), myeloma, mesothelioma, pancreatic cancer, Hodgkin's disease, testicular cancer, Waldenstrom's disease, head/neck cancer, cancer of the tongue, and/or malignancies induced by SV40 virus.

20. The method of claim 1, which is effective to achieve total remission (with no tumors or cancer blood markers detectable by any conventional cancer diagnostic method) within two-six months following onset of treatment, in at least 10-15% of stage IV cancer patients selected from one or more groups of patients diagnosed with Stage IV breast cancer, lung cancer, prostate cancer, skin cancer including melanoma, liver cancer, thyroid cancer, esophageal cancer, sarcoma, brain cancer of all types, colon and rectal cancers, bladder cancer, gall bladder cancer, stomach cancer, renal cancer, ovarian cancer, uterine cancer, cervical cancer, non-Hodgkin's lymphoma, acute myelogenous leukemia (AML), acute lymphocytic leukemia, chronic lymphocytic leukemia (CLL), myeloma, mesothelioma, pancreatic cancer, Hodgkin's disease, testicular cancer, Waldenstrom's disease, head/neck cancer, cancer of the tongue, and/or malignancies induced by SV₄₀ virus.

21. The method of claim 1, which is effective to achieve total remission (with no tumors or cancer blood markers detectable by any conventional cancer diagnostic method) within six months following onset of treatment, in at least 25% of stage IV cancer patients selected from one or more groups of patients diagnosed with treatment resistant Stage IV breast cancer, lung cancer, prostate cancer, skin cancer including melanoma, liver cancer, thyroid cancer, esophageal cancer, sarcoma, brain cancer of all types, colon and rectal cancers, bladder cancer, gall bladder cancer, stomach cancer, renal cancer, ovarian cancer, uterine cancer, cervical cancer, non-Hodgkin's lymphoma, acute myelogenous leukemia (AML), acute lymphocytic leukemia, chronic lymphocytic leukemia (CLL), myeloma, mesothelioma, pancreatic cancer, Hodgkin's disease, testicular cancer, Waldenstrom's disease, head/neck cancer, cancer of the tongue, and/or malignancies induced by SV₄₀ virus.

22. The method of claim 1, further comprising administration of a secondary anti-cancer or adjunctive therapeutic agent administered to said subject in a coordinate administration protocol, simultaneously with, prior to, or after administration of said cancer apoptosis-inducing glyco-benzaldehyde compound.

23. The method of claim 22, wherein the secondary anti-cancer or adjunctive therapeutic agent is selected from azacitidine, bevacizumab, bortezomib, capecitabine, cetuximab, clofarabine, dasatinib, decitabine, docetaxel, emend, erlotinib hydrochloride, exemestane, fulvestrant, gefitinib, gemcitabine hydrochloride, imatinib mesylate, imiquimod, lenalidomide, letrozole, nelarabine, oxaliplatin, paclitaxel, paclitaxel, docetaxel, palifermin, panitumumab, pegaspargase, pemetrexed disodium, rituximab, sorafenib tosylate, sunitinib malate, tamoxifen citrate, targretin, temozolomide, thalidomide, topotecan hydrochloride, trastuzumab, Bacillus Calmette-Guerin vaccine, interleukin-2, interferon α, rituximab, trastuzumab, filgrasten, G-CSF, epoetin alfa, erythropoietin, IL-11, oprelvekin, vorinostat, coenzyme q, palladium lipoic complexes, antineoplastins, cartilage, hydrazine sulfate, milk thistle, electrolytes, glutathione, alkaline water, grape seed extract, immunoglobulins, colostrum, oxidizing agents, glutathione, and mistletoe extract.

24. The method of claim 22, wherein the secondary anti-cellular proliferative agent or adjunctive therapeutic agent is alkaline water having a pH greater than 10.

25. The method of claim 22, wherein the secondary anti-cellular proliferative agent or adjunctive therapeutic agent is alkaline water having a pH greater of approximately 11 or higher.

26. A method for treating Stage IV cancer in a mammalian subject comprising administering an effective amount of a glyco-benzaldehyde compound sufficient to induce apoptosis in cancer cells in the subject, whereby cancer cells in the subject are destroyed in sufficient numbers to reduce or eliminate tumors and ablate circulating cancer cells in the subject over an effective treatment period and long-term survival of the subject is substantially prolonged.

27. The method of claim 1, which is effective to extend a five-year survival rate among Stage IV cancer patients (compared to median survival within a comparable group of conventionally-treated or untreated cancer patients) by at least 15%.

28. The method of claim 26, which is effective to extend a five-year survival rate among Stage IV cancer patients (compared to median survival within a comparable group of conventionally-treated or untreated cancer patients) by at least 25%.

29. The method of claim 26, which is effective to extend a five-year survival rate among Stage IV cancer patients (compared to median survival within a comparable group of conventionally-treated or untreated cancer patients) by 30-50% or greater.

30. The method of claim 26, which is effective to extend a five-year survival rate among Stage IV cancer patients diagnosed with a treatment-resistant cancer selected from treatment-resistant breast cancer, lung cancer, prostate cancer, skin cancer including melanoma, liver cancer, thyroid cancer, esophageal cancer, sarcoma, brain cancer of all types, colon and rectal cancers, bladder cancer, gall bladder cancer, stomach cancer, renal cancer, ovarian cancer, uterine cancer, cervical cancer, non-Hodgkin's lymphoma, acute myelogenous leukemia (AML), acute lymphocytic leukemia, chronic lymphocytic leukemia (CLL), myeloma, mesothelioma, pancreatic cancer, Hodgkin's disease, testicular cancer, Waldenstrom's disease, head/neck cancer, cancer of the tongue, and/or malignancies induced by SV₄₀ virus (compared to median survival within a comparable group of treatment-resistant cancer patients, receiving no further therapy or continuing to receive conventional oncotherapy) by at least 25%.

31. The method of claim 26, wherein the glyco-benzaldehyde compound is a compound of Formula I, below, or an analog of Formula I having the illustrated glycome moiety substituted by: 1) any α or β form hexose selected from mannose, galactose, or fructose; or 2) a biose formed from two or more hexoses wherein the hexoses may be the same or different.

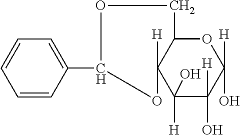

32. The method of claim 26, wherein the glyco-benzaldehyde compound is selected from 4, 6-0-benzylidine-D-glucopyranosyloxy, 2-β-D-glucopyranosyloxy benzaldehyde, 3-β-D-glucopyranosyloxy benzaldehyde, and 4-β-D-glucopyranosyloxy benzaldehyde.

33. The method of claim 26, wherein the glyco-benzaldehyde compound is administered to said subject in an anti-cancer effective dosage of from about 500 to about 4000 mg per day.

34. The method of claim 26, wherein the glyco-benzaldehyde compound is administered to said subject in an anti-cancer effective, intravenous (iv) dosage form comprising an aqueous solution of from about 2,000 to about 5,000 mg per day, wherein the subject is administered at least ten of these iv treatment doses within a first month of treatment.

35. The method of claim 26, wherein the glyco-benzaldehyde compound is administered to said subject in an anti-cancer effective, intravenous (iv) dosage form comprising an aqueous solution of from about 2,000 to about 5,000 mg per day, wherein the subject is administered 10-15 of these iv treatment doses within an initial three-week aggressive treatment period.

36. The method of claim 26, wherein the glyco-benzaldehyde compound is helicidum administered to said subject in an anti-cancer effective, intravenous (iv) dosage form comprising an aqueous solution of from about 2,000 to about 5,000 mg helicidum per day, wherein the subject is administered approximately 10-15 iv doses within an initial three week to one month aggressive treatment period, followed by maintenance treatment with an oral dosage form comprising about 1,000 to about 3,000 mg of helicidum formulated for oral delivery, per day.

37. The method of claim 26, which is effective to decrease tumor count in said subject by 10%-90% or greater over an effective course of treatment.

38. The method of claim 26, which is effective to decrease average tumor size in said subject by 10%-90% or greater over an effective course of treatment.

39. The method of claim 26, which is effective to decrease a circulating tumor cell (CTC) count in serial blood samples of said subject by 10%-90% or greater over an effective course of treatment.

40. The method of claim 26, which is effective to achieve total remission (with no tumors or cancer blood markers detectable by any conventional cancer diagnostic method) in at least 10-15% of stage IV cancer patients.

41. The method of claim 26, which is effective to treat one or more cancer forms selected from breast cancer, lung cancer, prostate cancer, skin cancer including melanoma, liver cancer, thyroid cancer, esophageal cancer, sarcoma, brain cancer of all types, colon and rectal cancers, bladder cancer, gall bladder cancer, stomach cancer, renal cancer, ovarian cancer, uterine cancer, cervical cancer, non-Hodgkin's lymphoma, acute myelogenous leukemia (AML), acute lymphocytic leukemia, chronic lymphocytic leukemia (CLL), myeloma, mesothelioma, pancreatic cancer, Hodgkin's disease, testicular cancer, Waldenstrom's disease, head/neck cancer, cancer of the tongue, and/or malignancies induced by SV40 virus.

42. The method of claim 26, which is effective to achieve total remission (with no tumors or cancer blood markers detectable by any conventional cancer diagnostic method) within six months following onset of treatment, in at least 10-15% of stage IV cancer patients from one or more groups of patients diagnosed with Stage IV breast cancer, lung cancer, prostate cancer, skin cancer including melanoma, liver cancer, thyroid cancer, esophageal cancer, sarcoma, brain cancer of all types, colon and rectal cancers, bladder cancer, gall bladder cancer, stomach cancer, renal cancer, ovarian cancer, uterine cancer, cervical cancer, non-Hodgkin's lymphoma, acute myelogenous leukemia (AML), acute lymphocytic leukemia, chronic lymphocytic leukemia (CLL), myeloma, mesothelioma, pancreatic cancer, Hodgkin's disease, testicular cancer, Waldenstrom's disease, head/neck cancer, cancer of the tongue, and/or malignancies induced by SV40 virus.

43. The method of claim 26, which is effective to achieve total remission (with no tumors or cancer blood markers detectable by any conventional cancer diagnostic method) within six months following onset of treatment, in at least 10-15% of stage IV cancer patients from one or more groups of patients diagnosed with treatment resistant Stage IV breast cancer, lung cancer, prostate cancer, skin cancer including melanoma, liver cancer, thyroid cancer, esophageal cancer, sarcoma, brain cancer of all types, colon and rectal cancers, bladder cancer, gall bladder cancer, stomach cancer, renal cancer, ovarian cancer, uterine cancer, cervical cancer, non-Hodgkin's lymphoma, acute myelogenous leukemia (AML), acute lymphocytic leukemia, chronic lymphocytic leukemia (CLL), myeloma, mesothelioma, pancreatic cancer, Hodgkin's disease, testicular cancer, Waldenstrom's disease, head/neck cancer, cancer of the tongue, and/or malignancies induced by SV₄₀ virus.

44. The method of claim 26, further comprising administration of a secondary anti-cancer or adjunctive therapeutic agent administered to said subject in a coordinate administration protocol, simultaneously with, prior to, or after administration of said glyco-benzaldehyde compound.

45. The method of claim 44, wherein the secondary anti-cancer or adjunctive therapeutic agent is selected from azacitidine, bevacizumab, bortezomib, capecitabine, cetuximab, clofarabine, dasatinib, decitabine, docetaxel, emend, erlotinib hydrochloride, exemestane, fulvestrant, gefitinib, gemcitabine hydrochloride, imatinib mesylate, imiquimod, lenalidomide, letrozole, nelarabine, oxaliplatin, paclitaxel, paclitaxel, docetaxel, palifermin, panitumumab, pegaspargase, pemetrexed disodium, rituximab, sorafenib tosylate, sunitinib malate, tamoxifen citrate, targretin, temozolomide, thalidomide, topotecan hydrochloride, trastuzumab, Bacillus Calmette-Guerin vaccine, interleukin-2, interferon α, rituximab, trastuzumab, filgrasten, G-CSF, epoetin alfa, erythropoietin, IL-11, oprelvekin, vorinostat, coenzyme q, palladium lipoic complexes, antineoplastins, cartilage, hydrazine sulfate, milk thistle, electrolytes, glutathione, alkaline water, grape seed extract, immunoglobulins, colostrum, oxidizing agents, glutathione, and mistletoe extract.

46. The method of claim 44, wherein the secondary anti-cellular proliferative agent or adjunctive therapeutic agent is alkaline water having a pH greater than 10.

47. The method of claim 44, wherein the secondary anti-cellular proliferative agent or adjunctive therapeutic agent is alkaline water having a pH greater of approximately 11 or higher.

48. An anti-cancer composition for preventing or alleviating cellular proliferative disorders in a mammalian subject comprising an apoptosis-inducing effective amount of a glyco-benzaldehyde combined with a secondary anti-cellular proliferative disorder or adjunctive therapeutic agent selected from the group consisting of azacitidine, bevacizumab, bortezomib, capecitabine, cetuximab, clofarabine, dasatinib, decitabine, docetaxel, emend, erlotinib hydrochloride, exemestane, fulvestrant, gefitinib, gemcitabine hydrochloride, imatinib mesylate, imiquimod, lenalidomide, letrozole, nelarabine, oxaliplatin, paclitaxel, paclitaxel, docetaxel, palifermin, panitumumab, pegaspargase, pemetrexed disodium, rituximab, sorafenib tosylate, sunitinib malate, tamoxifen citrate, targretin, temozolomide, thalidomide, topotecan hydrochloride, trastuzumab, Bacillus Calmette-Guerin vaccine, interleukin-2, interferon α, rituximab, trastuzumab, filgrasten, G-CSF, epoetin alfa, erythropoietin, IL-11, oprelvekin, vorinostat, coenzyme q, palladium lipoic complexes, antineoplastins, cartilage, hydrazine sulfate, milk thistle, electrolytes, glutathione, alkaline water, Poly-MVA®, grape seed extract, immunoglobulins, colostrum, oxidizing agents, and Dwarf mistletoe.

49. The anti-cancer composition of claim 49, wherein said apoptosis-inducing amount of said glyco-benzaldehyde comprises a daily dosage amount formulated for iv administration to said subject of from about 500 mg to about 4000 mg of said glyco-benzaldehyde.

50. A method for stimulating an immune response in a mammalian subject suffering from cancer or viral infection by reducing or eliminating a blood level of alpha-N-acetylgalactosaminidase (nagalase) in the subject, comprising administering to the subject an effective amount of a nagalase-disrupting glyco-benzaldehyde compound effective to reduce or ablate synthesis of nagalase in cancer cells and virus-infected cells, whereby circulating plasma levels of nagalase in the subject are reduced or eliminated and nagalase suppression of immune function in the subject is alleviated, thereby providing for stimulation of the immune response.

51. A method for stimulating a macrophage or natural killer (NK) cell-mediated immune response in a mammalian subject suffering from cancer or viral infection comprising administering to the subject an effective amount of a nagalase-disrupting glyco-benzaldehyde compound effective to reduce or ablate synthesis of nagalase in cancer cells and virus-infected cells, whereby circulating plasma levels of nagalase in the subject are reduced or eliminated and nagalase suppression of immune function in the subject is alleviated, thereby providing for stimulation of the macrophage or natural killer (NK) cell-mediated immune response.
